(19) United States
(12) Patent Application Publication
(10) Pub. No.: US 2015/0338425 A1
(43) Pub. Date: Nov. 26, 2015

(54) TREATMENT AND PROGNOSIS OF LYMPHANGIOLEIOMYOMATOSIS

(71) Applicants: THE BROAD INSTITUTE, INC., Cambridge, MA (US); THE BRIGHAM AND WOMEN'S HOSPITAL, INC., Boston, MA (US)

(72) Inventors: Elizabeth HENSKE, Cambridge, MA (US); CLISH Clary, Reading, MA (US); Carmen PRIOLO, Brookline, MA (US)

(73) Assignees: THE BRIGHAM AND WOMEN'S HOSPITAL, INC., Boston, MA (US); THE BROAD INSTITUTE, INC., Cambridge, MA (US)

(21) Appl. No.: 14/357,648

(22) PCT Filed: Nov. 14, 2012
(86) PCT No.: PCT/US12/64951

§ 371 Date: May 12, 2014

Related U.S. Application Data
(60) Provisional application No. 61/559,325, filed on Nov. 14, 2011.

(51) Int. Cl. G01N 33/92; A61K 51/00; A61K 31/517; A61K 31/4045; A61K 31/5377; A61K 31/513

(52) U.S. Cl. CPC G01N 33/92; A61K 31/5377; A61K 31/513; A61K 31/517; A61K 31/4045; A61K 51/00; G01N 2800/12

(57) ABSTRACT

Embodiments disclosed herein relate to treatment methods, diagnosis methods, drug efficacy evaluation methods and prognosis evaluation methods of lymphangioleiomyomatosis (LAM) and a disease associated with a mutation in a tuberous sclerosis complex (TSC) gene. The methods comprising analyses of the levels of lysophosphatidylcholine (LPC) in a biological sample of a subject, or comprising analyses of images of the location and signal intensity of isotope-labeled choline in the subject.

### CROSS REFERENCE TO RELATED APPLICATION

This application claims benefit under 35 U.S.C. §119(e) of U.S. Provisional Application No. 61/559,325 filed Nov. 14, 2011, the contents of which is incorporated herein by reference in its entirety.

### FIELD

The disclosure herein relates methods of treatment, diagnosis, disease activity monitoring, and testing drug efficacy of lymphangioleiomyomatosis (LAM) and a disease associated with a mutation in a tuberous sclerosis complex (TSC) gene.

### BACKGROUND

Lymphangioleiomyomatosis (LAM) is a multisystem disease occurring almost exclusively in women, usually presenting before menopause. The major organ affected is the lungs and the major system affected is lymphatics. LAM is characterized by the proliferation of abnormal smooth muscle-like cells (LAM cells) that can metastasize, leading to the formation of lung cysts and fluid-filled cystic structures in the axial lymphatics (e.g., lymphangioleiomyomas). In addition, the proliferation of LAM cells also produces angiomyolipomas which are benign tumors usually involving the kidneys. Angiomyolipomas have been found to comprise LAM cells and adipocytes, intermixed with incompletely developed vascular structures. The subsequent dilatation and obstruction can result in cystic collections of chylous material. Overdistension of these structures may cause them to rupture leading to the development of chylothorax or chylous ascites. Lymphangioleiomyomas may become sufficiently large to cause partial displacement of abdominal structures and chylous material has been observed to enter the pleural cavities apparently by rupture of the pleura or by transudation. Overall, the abnormal cell proliferation leads to progressive cystic destruction of the lung parenchyma, obstruction of lymphatics, airways, and progressive respiratory failure. A subject having LAM may present with progressive dyspnea, recurrent pneumothorax, chylothorax, or abdominal hemorrhage.

LAM occurs as a sporadic, non-heritable form (S-LAM) or in association with tuberous sclerosis complex (TSC-LAM), an autosomal dominant syndrome characterized by hamartoma-like tumor growths. The proliferating smooth muscle that occurs in the type of LAM seen in these patients with TSC-LAM has been shown to represent clones of the smooth muscle in those patients' renal angiomyolipomas. It is believed to represent metastases of this “benign” tumor.

The clinical course of patients with LAM can shows considerable variation. The disease can progressive slowly, but ultimately leads to respiratory failure and death. The 10-year survival from the start of symptoms has been reported to range from 47-79% depending on the various studies. There are currently no good treatment options for LAM. Current treatment used include administration of rapamycin (also known as sirolimus, an mTOR inhibitor) for shrinking tumors, and therapies targeting the reproductive cycle of the women, e.g., progesterone, oophorectomy, tamoxifen, gonadotropin-releasing hormone (GnRH) agonists or analogues and androgen therapy. The clinical course of LAM varies significantly, and there are no good predictors of clinical progression.

Tuberous sclerosis complex (TSC) is a rare multi-system genetic disease that results in non-malignant tumors to grow in the brain and on other vital organs such as the kidneys, heart, eyes, lungs, brain, and skin. A combination of symptoms may include seizures, developmental delay, behavioral problems, skin abnormalities, lungs and kidney disease. TSC is caused by a mutation of either of two genes, TSC1 and TSC2.

A high percentage (60-80%) of TSC patients has benign tumors in the kidneys called angiomyolipomas which frequently causing hematuria. These tumors are composed of vascular tissue (angio-), smooth muscle (-myo-), and fat (-lipoma). Although benign, angiomyolipomas may grow such that kidney function is impaired or the blood vessels may dilate and burst leading to catastrophic hemorrhage either spontaneously or with minimal trauma. Large angiomyolipomas can be treated with embolization.

Leading causes of death in TSC patients include renal disease, brain tumor, LAM of the lung, and status epilepticus or bronchopneumonia in those with severe mental handicap. There is no current effective treatment for TSC or the consequential AML or LAM; treatment is mainly symptomatic management, e.g., everolimus (derivative of rapamycin) for the treatment of subependymal giant cell astrocytoma (brain tumor), vigabatrin for infantile spasm, ACTH for epilepsy and rapamycin for shrinking the tumors.

Various methods are used to diagnose and monitor the status of LAM. For example, computed tomography scans (CT scans) are used to show thin-walled cysts scattered throughout the lungs, abdominal angiomyolipomas, and lymphangioleiomyomas. However, one drawback of a CT scan is that the cysts have to be big enough for visual detection. Often when this stage is reached, the subject has significant damage to the lung parenchyma, lymphatics, and airways and exhibit decrease pulmonary functions.

Various pulmonary function tests and exercise testing are also used to differentially diagnose and monitor the status of LAM. Pulmonary function tests can show reduced flow rates (FEV1) and diffusion capacity (DLCO). Exercise testing may reveal gas exchange abnormalities, ventilatory limitation, and hypoxemia, which can occur with near-normal lung function. Methods used to grade the severity of disease are the LAM histology score, semiquantitative and quantitative computer tomography, pulmonary function testing, and cardiopulmonary exercise testing. Currently, progression of disease is best assessed by serial measurements of FEV1, DL_{CO}, and exercise performance. However, the disadvantage of these physiologic testings is that often by the time there is detectable physiologic impairment, there is already significant damage within the subject. In addition, after imaging, pulmonary function tests and exercise testing indicate likely presence of the disease; lung biopsy is often required for confirmation.

Additional techniques that offer early diagnosis, earlier prediction of clinical progression that do not involve assessing pulmonary functions, and reducing or eliminating the need for lung biopsy for diagnosis and prognosis are needed in the management and treatment of this disease.

### SUMMARY

Embodiments of the disclosure are based on the discovery that the plasmas of human lymphangioleiomyomatosis (LAM) subjects have elevated lysophosphatidylcholine (LPC) species. Specifically, the levels of four LPC species (C16:0, C18:0, C18:1 and C20:4 LPC) were statistically increased over the corresponding LPCs levels of normal healthy subjects. In addition, LAM-derived cells have increased consumption of choline.

Accordingly, elevated levels of LPCs are useful in diagnosing LAM, both tuberous sclerosis complex lymphangioleiomyomatosis (TSC-LAM) and non-heritable sporadic form lymphangioleiomyomatosis (S-LAM), and also any disease associated with a mutation in a tuberous sclerosis complex (TSC) gene. The elevated levels of LPCs are useful treatment of the disease, useful for monitoring the progress of these disease conditions, and also useful for testing the effectiveness of therapeutic drugs and drug treatment of LAM or any disease associated with a mutation in a TSC gene.

Furthermore, the enzymes choline kinase (CK) and phospholipase A2 (PLA2) are therapeutic targets for the treatment of LAM or any disease associated with a mutation in a tuberous sclerosis complex (TSC) gene.

Accordingly, it is also the objective of this disclosure to provide methods for treating LAM, TSC-LAM and S-LAM, and also any disease associated with a mutation in a TSC gene.

It is the objective of this disclosure to provide methods for monitoring the disease activity of LAM, TSC-LAM and S-LAM, and also any disease associated with a mutation in a TSC gene. The methods for monitoring do not require assessing pulmonary functions which are only symptomatic when the disease has progress substantially. The methods for monitoring would also reduce or eliminate the need for lung biopsy.

In addition, it is the objective of this disclosure to provide methods for monitoring recurrence of LAM, TSC-LAM and S-LAM, and also any disease associated with a mutation in a TSC gene. The methods for monitoring do not require assessing pulmonary functions which are only symptomatic when the disease has progress substantially. The methods for monitoring would also reduce or eliminate the need for lung biopsy.

Furthermore, it is the objective of this disclosure to provide methods for assessing a drug efficacy in a subject having LAM, TSC-LAM and S-LAM, and also any disease associated with a mutation in a TSC gene.

Accordingly, in one embodiment, provided herein is a CK inhibitor and/or a PLA2 inhibitor for use in the treatment of LAM or a disease associated with a mutation in a TSC gene in a subject, wherein an elevated level of LPC in a biological sample from the subject is reduced.

In another embodiment, provided herein is a CK inhibitor and/or a PLA2 inhibitor for use in the manufacture of a medicament for the treatment of LAM or a disease associated with a mutation in a TSC gene in a subject, wherein an elevated level of LPC in a biological sample from the subject is reduced.

In one embodiment, provided herein is a method of treating LAM or a disease associated with a mutation in a TSC gene in a subject comprising administering a therapeutically effective amount of a CKinhibitor and/or a therapeutically effective amount of a PLA2 inhibitor to the subject in need thereof, wherein an elevated level of LPC in a biological sample from the subject is reduced or is at least 5% closer to a predetermined reference LPC level.

In one embodiment, provided herein is a method of treating LAM or a disease associated with a mutation in a TSC gene in a subject comprising diagnosing a subject as having LAM by a method comprising measuring the level of LPC in a biological sample obtained from the subject, wherein an elevated LPC level in the sample compared to a predetermined reference LPC level is indicative of LAM in the subject; and administering a therapeutically effective amount of a CK inhibitor and/or a therapeutically effective amount of a PLA2 inhibitor to the subject in need thereof, wherein an elevated level of LPC in a biological sample from the subject is reduced, or is at least 5% closer to a predetermined reference LPC level.

In one embodiment, provided herein is a method of treating LAM or a disease associated with a mutation in a TSC gene in a subject, the method comprising: measuring the level of LPC in a first biological sample obtained from the subject at a first time point; measuring the level of LPC in a second biological sample obtained from the subject at a second time point, wherein the first and second biological samples are of the same type, and wherein the first and second time are in chronological order; comparing the first measurement and the second measurement of LPC wherein the results of the comparison determines the course of LAM or the disease in the subject; deciding a course of action for the subject based upon the results of the comparison in the comparison step wherein the course of action is selected from the group consisting of maintained no treatment, terminating current treatment, modify current treatment, and maintained current treatment; and executing the course of action decided.

In one embodiment, provided herein is a method of treating LAM or a disease associated with a mutation in a TSC gene in a subject, the method comprising: administering to the subject a treatment for treating LAM or the disease; measuring the level of LPC in a first biological sample obtained from the subject at a first time point after start of the treatment; measuring the level of LPC in a second biological sample obtained from the subject at a second time point after start of treatment, wherein the first and second biological samples are of the same type, and wherein the first and second time are in chronological order; comparing the first measurement and the second measurement of LPC wherein the results of the comparison determines the course of LAM or disease in the subject; deciding a course of action for the subject based upon the results of the comparison in the comparison step wherein the course of action is selected from the group consisting of terminating current treatment, modify current treatment, and maintained current treatment; and executing the course of action decided.

In one embodiment, provided herein is a method of treating LAM or a disease associated with a mutation in a TSC gene in a subject, the method comprising: measuring the level of LPC in a first biological sample obtained from the subject at a first time point prior to start of a treatment; administering to the subject a treatment for treating LAM or the disease; measuring the level of LPC in a second biological sample obtained from the subject at a second time point after start of the treatment, wherein the first and second biological samples are of the same type, and wherein the first and second time are in chronological order; comparing the first measurement and the second measurement of LPC wherein the results of the comparison determines the course of LAM or disease in the subject; deciding a course of action for the subject based upon the results of the comparison in the comparison step wherein the course of action is selected from the group consisting of terminating current treatment, modify current treatment, and maintained current treatment; and executing the course of action decided.

In one embodiment, provided herein is a method of assessing the efficacy of a drug treatment in a subject, the method comprising: administering to the subject a treatment, wherein the subject has LAM or a disease associated with a mutation in a TSC gene; measuring the level of LPC in a first biological sample obtained from the subject at a first time point; measuring the level of LPC in a second biological sample obtained from the subject at a second time point, wherein the first and second biological samples are of the same type, and wherein the first and second time are in chronological order; comparing the first measurement and the second measurement of LPC wherein the results of the comparison determines whether the drug treatment is effective in the subject; and deciding a course of action for the subject based upon the results of the comparison in the comparison step wherein the course of action is selected from the group consisting of maintained current drug application, terminating current drug application, and modify current drug application.

In one embodiment, provided herein is a method of assessing the efficacy of a drug treatment in a subject, the method comprising: measuring the level of LPC in a first biological sample obtained from the subject at a first time point, wherein the subject has LAM or a disease associated with a mutation in a TSC gene; administering to the subject a treatment; measuring the level of LPC in a second biological sample obtained from the subject at a second time point, wherein the first and second biological samples are of the same type, and wherein the first and second time are in chronological order; comparing the first measurement and the second measurement of LPC wherein the results of the comparison determines whether the drug treatment is effective in the subject; and deciding a course of action for the subject based upon the results of the comparison in the comparison step wherein the course of action is selected from the group consisting of maintained current drug application, terminating current drug application, and modify current drug application.

In one embodiment, provided herein is a method of treating LAM or a disease associated with a mutation in a TSC gene in a subject, the method comprising: imaging the density and location of carbon-11-labeled choline, fluorine 18-labeled choline or derivatives thereof in the subject at a first time; imaging the density and location of carbon-11-labeled choline, fluorine 18-labeled choline or derivatives thereof in the subject at a second time, wherein the first and second time are in chronological order; comparing the first imaging and the second imaging, wherein the results of the comparison determines the course of LAM or disease in the subject; deciding a course of action for the subject based upon the results of the comparison in the comparison step wherein the course of action is selected from the group consisting of maintained no treatment, terminating current treatment, modify current treatment, and maintained current treatment; and executing the course of action decided.

A method of treating LAM or a disease associated with a mutation in a TSC gene in a subject, the method comprising: administering to the subject a treatment; imaging the density and location of carbon-11-labeled choline, fluorine 18-labeled choline or derivatives thereof in the subject at a first time; imaging the density and location of carbon-11-labeled choline, fluorine 18-labeled choline or derivatives thereof in the subject at a second time, wherein the first and second time are in chronological order; comparing the first imaging and the second imaging, wherein the results of the comparison determines the course of LAM or disease in the subject; deciding a course of action for the subject based upon the results of the comparison in the comparison step wherein the course of action is selected from the group consisting of terminating current treatment, modify current treatment, and maintained current treatment; and executing the course of action decided.

In one embodiment, provided herein is a method of treating LAM or a disease associated with a mutation in a TSC gene in a subject, the method comprising: imaging the density and location of carbon-11-labeled choline, fluorine 18-labeled choline or derivatives thereof in the subject at a first time; administering to the subject a treatment; imaging the density and location of carbon-11-labeled choline, fluorine 18-labeled choline or derivatives thereof in the subject at a second time, wherein the first and second time are in chronological order; comparing the first imaging and the second imaging, wherein the results of the comparison determines the course of LAM or disease in the subject; deciding a course of action for the subject based upon the results of the comparison in the comparison step wherein the course of action is selected from the group consisting of terminating current treatment, modify current treatment, and maintained current treatment; and executing the course of action decided.

In one embodiment of any methods described herein, the subject has been previously diagnosed as having elevated LPC levels over a predetermined reference LPC level.

In one embodiment of any methods described herein, the reduction in LPC level is at least 5% compared to the previously diagnosed elevated LPC level.

In one embodiment of any methods described herein, the reduction in LPC level is at least 5% compared to the predetermined reference LPC level.

In one embodiment of any methods described herein, the predetermined reference LPC level comprises the average LPC level measurements from a plurality of healthy subjects.

In one embodiment of any methods described herein, the method further comprising selecting a subject having LAM or a disease associated with a mutation in a TSC gene.

In one embodiment of any methods described herein, the method further comprising determining that the subject having LAM has elevated LPC level in a biological sample obtained from the subject.

In one embodiment of any methods described herein, the mutation is in the TSC1 gene and/or TSC 2 gene.

In one embodiment of any methods described herein, the mutation results in no protein production or no functional protein.

In one embodiment of any methods described herein, the disease is LAM.

In one embodiment of any methods described herein, the LAM is selected from the group consisting of tuberous sclerosis complex lymphangioleiomyomatosis (TSC-LAM) and non-heritable sporadic form lymphangioleiomyomatosis (S-LAM).

In one embodiment of any methods described herein, the predetermined reference LPC level comprises an average LPC level measurement from a plurality of healthy subjects without LAM or any or disease associated with a mutation in a TSC gene, wherein an elevated level of LPC in the sample compared to the predetermined reference LPC level is diagnostic of the presence of LAM or the disease associated with a mutation in a TSC gene respectively.

In one embodiment of any methods described herein, the subject is female.

In one embodiment of any methods described herein, the female is of reproductive age.

In one embodiment of any methods described herein, the treatment is selected from the group consisting of hormone therapy, CK inhibition therapy, PLA2 inhibition therapy, mTORC1 inhibition therapy, PI3K inhibition therapy, oxygen therapy, pleurodesis, embolization, ablation or resection of angiomyolipomas; bronchodilator therapy, withdrawal from estrogen-containing medications, and thoracic duct ligation.

In one embodiment of any methods described herein, the treatment is selected from the group consisting of hormone therapy, CK inhibition therapy, PLA2 inhibition therapy, mTORC1 inhibition therapy, and PI3K inhibition therapy.

In one embodiment of any methods described herein, the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, PI-103 and cyclophane.

In one embodiment of any methods described herein, the PLA2 inhibitor is selected from the group consisting of darapladib, bromoenol lactone, varespladib and palmitoyl trifluoromethyl ketone.

In one embodiment of any methods described herein, the PI3K inhibitor is selected from the group consisting of perifosine, wortmannin, demethoxyviridin, LY294002, CAL101, PX-866, BEZ235, SF1126, INK1117, IPI-145, GDC-0941, BKM120, XL147, XL765, Palomid 529, GSK1059615, ZSTK474, PWT33597, IC87114, TG100-115, CAL263, PI-103, GNE-477, CUDC-907, and AEZS-136.

In one embodiment of any methods described herein, the mTORC1 inhibitor is selected from the group consisting of everolimus, temsirolimus and sirolimus.

In one embodiment of any methods described herein, the LPC is measured by liquid chromatography coupled to mass spectrometry (LC-MS), enzymatic measurements, or gas chromatography coupled to chemical ionization mass spectrometry (GC-CIMS).

In one embodiment of any methods described herein, the LPC measured is selected from the selected from the group consisting of: C14:0 LPC; C16:0 LPC; C16:1 LPC; C18:0 LPC; C18.1 LPC; C18:3 LPC; C18:2 LPC; C20:3 LPC; C20:4 LPC; and C22:6 LPC.

In one embodiment of any methods described herein, the biological samples are serum, plasma or urine.

In one embodiment of any methods described herein, the first time point is selected for the group consisting of prior to an application of a treatment, during the course of a first treatment, the course of a second treatment, and the course of a subsequent treatment.

In one embodiment of any methods described herein, the second time point is selected for the group consisting of prior to an application of a treatment, during the course of a first treatment, the course of a second treatment, and the course of a subsequent treatment.

In one embodiment of any methods described herein, course of LAM or the disease in the subject in selected from the group consisting of no further progression, continued progression and regression.

In one embodiment of any methods described herein, both the first and second measurements of LPC are prior to an application of a treatment, and wherein the second measurement of LPC is the same or is no more than 5% over the first measurement of LPC indicates that there is no further progression of LAM in the subject, and the course of action is to maintain no treatment in the subject.

In one embodiment of any methods described herein, both the first and second measurements of LPC are prior to an application of a treatment, and wherein the second measurement of LPC is at least 5% over or higher the first measurement of LPC indicates that there is progression of LAM in the subject, and the course of action is to initiate a treatment in the subject.

In one embodiment of any methods described herein, both the first and second measurements of LPC are during the course of a first or second or subsequent treatment, wherein the second measurement of LPC is during the course of the treatment is at least lower by 5% compared to the first measurement of LPC indicates regression of LAM in the subject, and that the current treatment is effective and is to be maintained.

In one embodiment of any methods described herein, both the first and second measurements of LPC are during the course of a first or second or subsequent treatment, wherein the second measurement of LPC is during the course of the treatment remains the same as or is higher by at least 5% over the first measurement of LPC indicates continued progression of LAM in the subject, and that the current treatment is ineffective and is to be terminated or modified.

In one embodiment of any methods described herein, the first measurement of LPC is prior to an application of a treatment and the second measurement of LPC is during the course of a first or second or subsequent treatment, wherein the second measurement of LPC during the course of the treatment is the same as or elevated at least 5% over or higher the first measurement of LPC prior to start of the treatment indicates continued progression of LAM in the subject, and that the current treatment is ineffective and is to be terminated or modified.

In one embodiment of any methods described herein, the first measurement of LPC is prior to an application of a treatment and the second measurement of LPC is during the course of the first or second or subsequent treatment, wherein the second measurement of LPC is at least lower by 5% compared to the first measurement of LPC indicates regression of LAM in the subject, and that the current treatment is effective and is to be maintained.

In one embodiment of any methods described herein, the first measurement of LPC is a first treatment and the second measurement of LPC is during the course of the second or subsequent treatment, wherein the second measurement of LPC is the same as or elevated at least 5% over the first measurement of LPC indicates continued progression of LAM in the subject, and that the second or subsequent treatment is ineffective and is to be terminated or modified.

In one embodiment of any methods described herein, the first measurement of LPC is a first treatment and the second measurement of LPC is during the course of the second or subsequent treatment, wherein the second measurement of LPC is at least lower by 5% compared to the first measurement of LPC indicates regression of LAM in the subject, and that the second or subsequent treatment is effective and is to be maintained.

In one embodiment of any methods described herein, the second imaging has lower density and location signal by at least 5% compared to the first imaging indicates regression of LAM in the subject, and that the course of action is to be maintained.

In one embodiment of any methods described herein, the second imaging density and location signals remain the same as or are higher by at least 5% over the first imaging indicates progression of LAM in the subject, and that the course of action is to be terminated or modified.

### DEFINITIONS

As used herein, the term “comprising” or “comprises” is used in reference to methods and respective component(s) thereof, that are essential to the claims, yet open to the inclusion of unspecified elements, whether essential or not. The use of “comprising” indicates inclusion rather than limitation.

The term “consisting of” refers to methods and respective components thereof as described herein, which are exclusive of any element not recited in that description of the embodiment.

A “cancer” in a subject refers to the presence of cells possessing characteristics typical of cancer-causing cells, such as uncontrolled proliferation, immortality, metastatic potential, rapid growth and proliferation rate, loss of contact inhibition and certain characteristic morphological features. Often, cancer cells will be in the form of a tumor, but such cells may exist alone within a subject, or may be a non-tumorigenic cancer cell, such as a leukemia cell. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease the natural course of which is fatal. Cancer cells exhibit the properties of invasion and metastasis and are highly anaplastic.

In one embodiment, “administration,” “treating,” and “treatment,” as it applies to a subject, refers to the contact of an exogenous pharmaceutical, a drug, a compound, a therapeutic, or a composition to the subject. In another embodiment, “administration,” “treating,” and “treatment,” as it applies to a subject, refers to the contact of any one of the described inhibitor to the subject.

Alternatively, the terms “administering,” refers to the placement of any inhibitor or treatment described herein for intended purposes such as treating LAM cells, cancer cells, tumor cells, tumor cells with TSC mutation(s) and/or mTOR hyperactivity in the subject. The compound, the inhibitor or treatment described herein can be administered by any appropriate route which results in an effective treatment in the subject, i.e. administration results in delivery to a desired location (e.g., directly to a tumor or near a tumor) in the subject where at least a portion of the composition delivered. The period of time the inhibitor, the combination of inhibitors is active depends on the half-life in vivo after administration to a subject, and can be as short as a few hours, e. g. twenty-four hours, to a few days, to as long as several years. Modes of administration include injection, infusion, instillation, suppository (e.g., for vaginal, cervical. rectal or urethral insertion), percutaneous implantation or ingestion. “Injection” includes, without limitation, intravenous, intramuscular, intraarterial, intraventricular, intradermal, intraperitoneal, subcutaneous, subcuticular injection and infusion.

In one embodiment, as used herein, the term “treat′ or treatment” refers to reducing or alleviating at least one adverse clinical symptom associated with cancer, e.g., pain, swelling, low blood count etc. In another embodiment, the term “treat′ or treatment” refers to slowing or reversing the progression neoplastic uncontrolled cell multiplication, i.e. shrinking existing tumors and/or halting tumor growth. In another embodiment, the term “treat′ or treatment” refers to inducing apoptosis in LAM or tumor cells in the subject.

As used herein, the term “a therapeutically effective amount” or “an effective amount” refers to an amount sufficient to achieve the intended purposes such as treating LAM cells, cancer cells, tumor cells, tumor cells with TSC mutation(s) and/or mTOR hyperactivity. In one embodiment, a therapeutically effective amount of an inhibitor, a combination of inhibitors, or a composition described herein for a method of treating cancer or TSC is an amount of sufficient to induce apoptosis of cancer cells of the subject as compared to in the absent of the an inhibitor, the combination of inhibitors, a composition respectively. In other embodiments, the amount that is safe and sufficient to treat, delay the development of a tumor, and/or delay further growth of the tumor. In some embodiments, the amount can thus cure or result in amelioration of the symptoms of cancer and tumor growth, slow the course of cancer progression, slow or inhibit a symptom of cancer, slow or inhibit the establishment of secondary symptoms of cancer or inhibit the development of a secondary symptom of the cancer. For example, an effective amount of an inhibitor, a combination of inhibitors, or a composition described herein can inhibits tumor (e.g., LAM or AML) further growth, cause a reduction in size or even completely halt tumor growth, shrink the sizes of tumor, even complete regression of tumor, and reduce clinical symptoms associated with tumor. In one embodiment, an effective amount for treating cancer or TSC is an amount of an inhibitor, a combination of inhibitors, or a composition described herein sufficient to result in a reduction or complete removal of the symptoms of the disorder, disease, or medical condition. In another embodiment, an effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount”. However, for any given case, an appropriate “effective amount” can be determined by a skilled artisan according to established methods in the art using only routine experimentation.

Derivatives, as used herein, include a chemically modified compound wherein the modification is considered routine by the ordinary skilled chemist, such as additional chemical moieties (e.g., an ester or an amide of an acid, protecting groups, such as a benzyl group for an alcohol or thiol, and tert-butoxycarbonyl group for an amine). Derivatives also include radioactively labeled of the compounds described herein (e.g., biotin or avidin, with enzymes such as horseradish peroxidase and the like, with bioluminescent agents, chemoluminescent agents or fluorescent agents). Additionally, moieties may be added to the compounds described herein or a portion thereof to increase half-life in vivo. Derivatives, as used herein, also encompasses analogs, such as a compound that comprises a chemically modified form of a specific compound or class thereof, and that maintains the pharmaceutical and/or pharmacological activities characteristic of said compound or class, are also encompassed in the present invention. In one embodiment, derivatives, as used herein, also encompasses prodrugs of the compounds described herein, which are known to enhance numerous desirable qualities of pharmaceuticals (e.g., solubility, bioavailability, manufacturing, etc.).

As used herein, the term “mTOR deregulation” with respect to cancer cells or cells with neoplasia refers to increased or decreased signaling of the mTOR pathway compared to normal cells or cells without neoplasia. Increased or decreased signaling can be analyzed by any method known in the art, e.g., by monitoring the corresponding increase or decrease phosphorylation of the mTOR downstream effectors molecules S6K1 and 4E-BP1. See L. Yan, 2006 J. Biol. Chem., 281: 19793-19797.

As used herein, the term “mTOR hyperactivation” with respect to cancer cells or cells with neoplasia refers to increased signaling of the mTOR pathway compared to normal cells or cells without neoplasia. Increased mTOR signaling can be analyzed by any method known in the art, e.g., by monitoring the increase phosphorylation of the mTOR downstream effectors molecules S6K1 and 4E-BP1. See L. Yan, 2006 J. Biol. Chem., 281: 19793-19797.

The term “gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence. A gene may also be an mRNA or cDNA corresponding to the coding regions (e.g., exons and miRNA) optionally comprising 5′- or 3′-untranslated sequences linked thereto. In one embodiment, a gene may also be an amplified nucleic acid molecule produced in vitro comprising all or a part of the coding region and/or 5′- or 3′-untranslated or regulatory sequences linked thereto.

As used herein, the term “recurrence” of LAM or a disease associated with a mutation in a TSC gene refers to the re-manifestation/re-development of known symptoms associated with the LAM or the disease after previous successful treatment.

As used herein, the term “prognosis” encompasses predictions and likelihood analysis of LAM or likelihood of the disease progression, particularly LAM recurrence, metastatic spread, and disease recurrence. The prognosis method described herein is intended for clinical use in making decision concerning treatment modalities, including therapeutic interventions, diagnostic criteria such as disease staging, and disease monitoring and surveillance for metastasis or recurrence of neoplastic disease.

### BRIEF DESCRIPTION OF THE DRAWINGS

FIG. 1. Lysophosphatidylcholine (LPC) and collagen component levels are significantly higher in LAM plasma specimens than healthy controls. Plasma from LAM (n=14) and healthy control (n=9) were subject to metabolomic profiling. LPC species and the collagen components glycine and hydroxyproline were significantly higher in LAM patients than healthy controls.* p<0.05.

FIG. 2. Schematic diagram summarizing the choline phospholipid metabolism.

FIG. 3. Heat map of all differential metabolites in ELT3 (V3) cells vs. isogenic control re-expressing TSC2 (T3). Peak areas were normalized to cellular protein content, and range scaling of each feature across samples was computed. Sixty-one out of 248 metabolites measured were significant (q<0.05). The zoomed view shows 10 choline metabolism-related compounds.

FIG. 4. Choline consumption in TSC2-deficient cells. Levels of choline were measured by LC-MS in the culture media of ELT3-V3 (TSC2-deficient), ELT3-T3 (re-expressing TSC2) and patient-derived 621-101 cells. V3 cells showed higher consumption of choline compared to control (T3). Rapamycin (Rapa) treatment decreased choline consumption in 621-101 cells (statistics as in FIG. 3; *q<0.05).

FIG. 5 is a block diagram showing an exemplary system for assessing disease activity and assessing drug treatment efficacy for LAM, prognosis, and relapse survelliance.

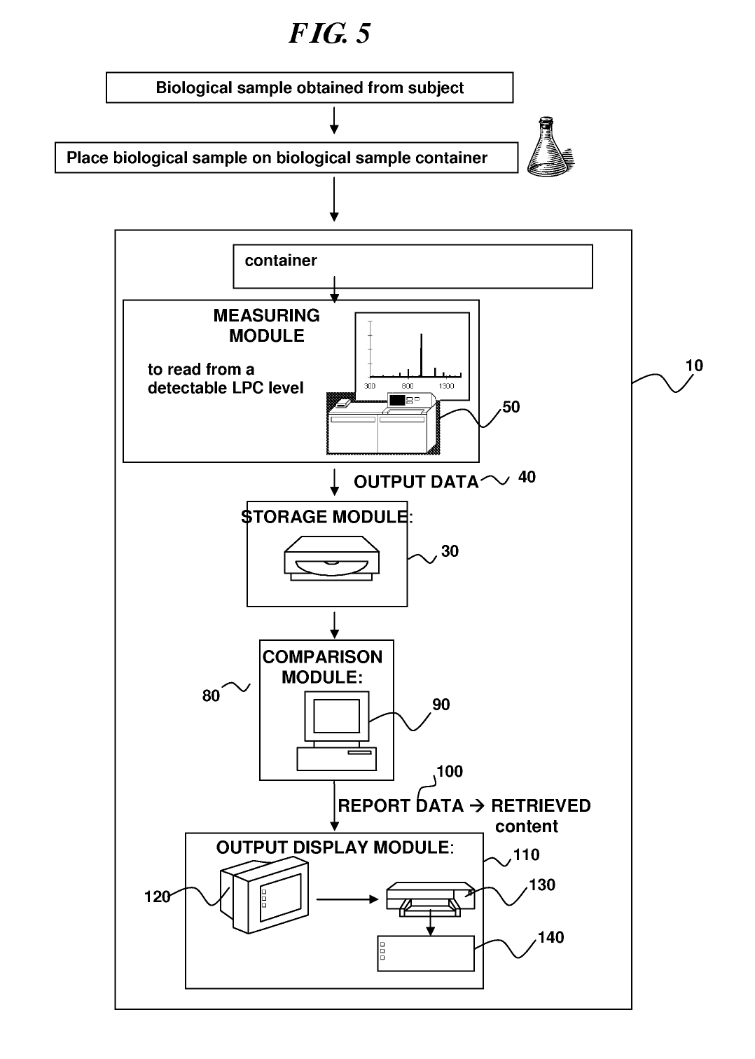

FIG. 6 is an exemplary set of instructions on a computer readable storage medium for use with the systems described herein.

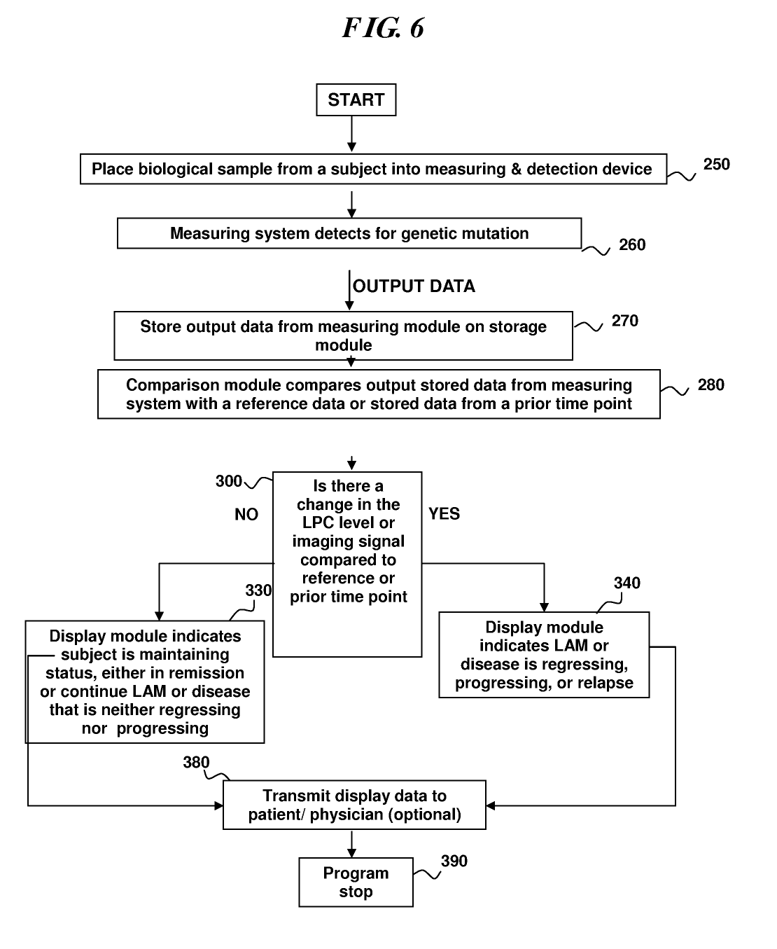

### DETAILED DESCRIPTION

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Meolcular Biology, Vo. 203, 2003, Transgenic Mouse, editored by Marten H. Hofker and Jan van Deursen, which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments of the disclosure are based on the discovery that the plasmas of human lymphangioleiomyomatosis (LAM) subjects have elevated lysophosphatidylcholine (LPC) species. Specifically, the levels of four LPC species (C16:0, C18:0, C18:1 and C20:4 LPC) were statistically increased over the LPCs levels of normal healthy subjects. In addition, LAM-derived cells have increased consumption of choline.

Accordingly, elevated levels of LPCs are useful in diagnosing LAM, both tuberous sclerosis complex lymphangioleiomyomatosis (TSC-LAM) and non-heritable sporadic form lymphangioleiomyomatosis (S-LAM), and also any disease associated with a mutation in a tuberous sclerosis complex (TSC) gene.

Elevated levels of LPCs are also useful for monitoring the progress of these disease conditions; if the LPC levels remains elevated or continue to rise, then these diseases are progressing in the natural course of these diseases and would likely cause deterioration of physiologic and pulmonary functions in the afflicted subjects. On the other hand, if the LPC levels decreased from the original elevated levels and the decreased levels are getting closer to that of normal healthy subjects, then these diseases are not progressing. These disease conditions can be considered to be regressing. When the LPC levels remained more or less constant at the original elevated levels, these disease conditions can be considered to be holding status quo, and is neither progressing nor regressing.

Elevated levels of LPCs are also useful for testing the effectiveness of therapeutic drugs that are used in the treatment of LAM, TSC-LAM and S-LAM, and also any disease associated with a mutation in a TSC gene. A therapeutic drug is effective if the LPC levels in the treated subject decreased from the original elevated levels and the decreased levels are getting closer to that of normal healthy subjects, or if the LPC levels remained more or less constant at the original elevated levels, ie, no further elevation from the original elevated levels. A therapeutic drug is ineffective if the LPC levels remains elevated or continue to rise over the original elevated levels.

Furthermore, the presence of elevated levels of LPCs in these disease conditions indicates that there is at least a dysfunction in the key enzymes of the phospholipid pathway. Since the defects in the rapamycin (mTOR) signalling pathway result in a loss of control in cell growth and cell division, and a mutation in at least one of the two TSC gene consequently leads of the deregulation of the mTOR signalling pathway, degradation of the mTOR signalling pathway contributes to the elevated levels of LPCs in these disease conditions. The identified key enzymes are choline kinase (CK), phospholipase A2 (PLA2), and phosphatidylinositol 3-kinase (PI 3-kinases or PI3Ks) and they are over active. Accordingly, these key enzymes present targets for therapeutic intervention and drug discovery.

Accordingly, in one embodiment, provided herein is a method of monitoring the disease activity of LAM, TSC-LAM and S-LAM, or a disease associated with a mutation in a TSC gene a subject, the method comprising: (a) measuring the level of LPC in a first biological sample obtained from the subject at a first time point; (b) measuring the level of LPC in a second biological sample obtained from the subject at a second time point, wherein the first and second biological samples are of the same type, and wherein the first and second time are in chronological order; (c) comparing the first measurement and the second measurement of LPC wherein the results of the comparison determines the course of LAM or the disease in the subject; and (d) deciding a course of action for the subject based upon the results of the comparison in step (c) wherein the course of action is selected from the group consisting of maintained no treatment, terminating current treatment, modify current treatment, and maintained current treatment.

In one embodiment, provided herein is a method of treating LAM, TSC-LAM and S-LAM, or a disease associated with a mutation in a TSC gene a subject, the method comprising: (a) measuring the level of LPC in a first biological sample obtained from the subject at a first time point; (b) measuring the level of LPC in a second biological sample obtained from the subject at a second time point, wherein the first and second biological samples are of the same type, and wherein the first and second time are in chronological order; (c) comparing the first measurement and the second measurement of LPC wherein the results of the comparison determines the course of LAM or the disease in the subject; (d) deciding a course of action for the subject based upon the results of the comparison in step (c) wherein the course of action is selected from the group consisting of maintained no treatment, terminating current treatment, modify current treatment, and maintained current treatment; and (e) executing the course of action. In one embodiment of this treatment method, the method further comprises administering a treatment to the subject for treating the disease.

In one embodiment of any method described herein, the first measurement and the second measurement of LPC are compared with a predetermined reference LPC level, wherein the results of the comparison determines the course of LAM or disease condition in the subject. On the basis of the results of the comparison, the physician can decide a course of action for the subject, e.g., maintain no treatment regimen or a treatment plan selected from the group consisting of no treatment regimen, terminating current treatment, modify current treatment, or maintained current treatment.

Accordingly, in one embodiment, provided herein is a method of monitoring the disease activity of LAM, TSC-LAM and S-LAM, or a disease associated with a mutation in a TSC gene a subject, the method comprising: (a) measuring the level of LPC in a first biological sample obtained from the subject at a first time point; (b) measuring the level of LPC in a second biological sample obtained from the subject at a second time, wherein the first and second biological samples are of the same type, and wherein the first and second time are in chronological order; (c) comparing the first measurement and the second measurement of LPC with a predetermined reference LPC level, wherein the results of the comparison determines the course of LAM or the disease in the subject; and (d) deciding a course of action for the subject selected from the group consisting of terminating current treatment, modify current treatment, or maintained current treatment.

In another embodiment, provided herein is a method of treating LAM, TSC-LAM and S-LAM, or a disease associated with a mutation in a TSC gene a subject, the method comprising: (a) measuring the level of LPC in a first biological sample obtained from the subject at a first time point; (b) measuring the level of LPC in a second biological sample obtained from the subject at a second time, wherein the first and second biological samples are of the same type, and wherein the first and second time are in chronological order; (c) comparing the first measurement and the second measurement of LPC with a predetermined reference LPC level, wherein the results of the comparison determines the course of LAM or the disease in the subject; (d) deciding a course of action for the subject selected from the group consisting of terminating current treatment, modify current treatment, or maintained current treatment; and (e) executing the course of action. In one embodiment of this treatment method, the method further comprises administering a treatment to the subject for treating the disease.

In one embodiment, provided herein is a method of monitoring the disease activity of LAM, TSC-LAM and S-LAM, or a disease associated with a mutation in a TSC gene a subject, the method comprising: (a) administering to the subject a treatment; (b) measuring the level of LPC in a first biological sample obtained from the subject at a first time point after start of the treatment; (c) measuring the level of LPC in a second biological sample obtained from the subject at a second time point after start of treatment, wherein the first and second biological samples are of the same type, and wherein the first and second time are in chronological order; (d) comparing the first measurement and the second measurement of LPC wherein the results of the comparison determines the course of LAM or the disease in the subject; and (e) deciding a course of action for the subject based upon the results of the comparison in step (d) wherein the course of action is selected from the group consisting of terminating current treatment, modify current treatment, and maintained current treatment.

In one embodiment, provided herein is a method of monitoring the disease activity of LAM, TSC-LAM and S-LAM, or a disease associated with a mutation in a TSC gene a subject, the method comprising: (a) administering to the subject a treatment, wherein the treatment is selected from the group consisting of hormone therapy, choline kinase inhibition therapy, phospholipase A2 (PLA2) inhibitor therapy, mTORC1 inhibitor therapy, and phosphatidylinositol 3-kinase (PI 3-kinases or PI3Ks) inhibitor therapy; (b) measuring the level of LPC in a first biological sample obtained from the subject at a first time point after start of the treatment; (c) measuring the level of LPC in a second biological sample obtained from the subject at a second time point after start of treatment, wherein the first and second biological samples are of the same type, and wherein the first and second time are in chronological order; (d) comparing the first measurement and the second measurement of LPC wherein the results of the comparison determines the course of LAM or the disease in the subject; and (e) deciding a course of action for the subject based upon the results of the comparison in step (d) wherein the course of action is selected from the group consisting of terminating current treatment, modify current treatment, and maintained current treatment.

In another embodiment, provided herein is a method of treating LAM, TSC-LAM and S-LAM, or a disease associated with a mutation in a TSC gene a subject, the method comprising: (a) administering to the subject a treatment; (b) measuring the level of LPC in a first biological sample obtained from the subject at a first time point after start of the treatment; (c) measuring the level of LPC in a second biological sample obtained from the subject at a second time point after start of treatment, wherein the first and second biological samples are of the same type, and wherein the first and second time are in chronological order; (d) comparing the first measurement and the second measurement of LPC wherein the results of the comparison determines the course of LAM or the disease in the subject; (e) deciding a course of action for the subject based upon the results of the comparison in step (d) wherein the course of action is selected from the group consisting of terminating current treatment, modify current treatment, and maintained current treatment; and (f) executing the course of action. In one embodiment of this treatment method, the method further comprises administering an additional or new treatment to the subject for treating the disease.

In another embodiment, provided herein is a method of treating LAM, TSC-LAM and S-LAM, or a disease associated with a mutation in a TSC gene a subject, the method comprising: (a) administering to the subject a treatment, wherein the treatment is selected from the group consisting of hormone therapy, CK inhibition therapy, PLA2 inhibition therapy, mTORC1 inhibition therapy, and PI3Ks inhibition therapy; (b) measuring the level of LPC in a first biological sample obtained from the subject at a first time point after start of the treatment; (c) measuring the level of LPC in a second biological sample obtained from the subject at a second time point after start of treatment, wherein the first and second biological samples are of the same type, and wherein the first and second time are in chronological order; (d) comparing the first measurement and the second measurement of LPC wherein the results of the comparison determines the course of LAM or the disease in the subject; (e) deciding a course of action for the subject based upon the results of the comparison in step (d) wherein the course of action is selected from the group consisting of terminating current treatment, modify current treatment, and maintained current treatment; and (f) executing the course of action. In one embodiment of this treatment method, the method further comprises administering an additional or new treatment to the subject for treating the disease.

In one embodiment, provided herein is a method of monitoring the disease activity of LAM, TSC-LAM and S-LAM, or a disease associated with a mutation in a TSC gene a subject, the method comprising: (a) measuring the level of LPC in a first biological sample obtained from the subject at a first time point prior to start of a treatment; (b) administering to the subject a treatment; (c) measuring the level of LPC in a second biological sample obtained from the subject at a second time point after start of the treatment, wherein the first and second biological samples are of the same type, and wherein the first and second time are in chronological order; (d) comparing the first measurement and the second measurement of LPC wherein the results of the comparison determines the course of LAM or the disease in the subject; and (e) deciding a course of action for the subject based upon the results of the comparison in step (d) wherein the course of action is selected from the group consisting of terminating current treatment, modify current treatment, and maintained current treatment.

In one embodiment, provided herein is a method of monitoring the disease activity of LAM, TSC-LAM and S-LAM, or a disease associated with a mutation in a TSC gene a subject, the method comprising: (a) measuring the level of LPC in a first biological sample obtained from the subject at a first time point prior to start of a treatment; (b) administering to the subject a treatment, wherein the treatment is selected from the group consisting of hormone therapy, CK inhibition therapy, PLA2 inhibition therapy, mTORC1 inhibitor therapy, and PI3Ks inhibition therapy; (c) measuring the level of LPC in a second biological sample obtained from the subject at a second time point after start of the treatment, wherein the first and second biological samples are of the same type, and wherein the first and second time are in chronological order; (d) comparing the first measurement and the second measurement of LPC wherein the results of the comparison determines the course of LAM or the disease in the subject; and (e) deciding a course of action for the subject based upon the results of the comparison in step (d) wherein the course of action is selected from the group consisting of terminating current treatment, modify current treatment, and maintained current treatment.

In another embodiment, provided herein is a method of treating LAM, TSC-LAM and S-LAM, or a disease associated with a mutation in a TSC gene a subject, the method comprising: (a) measuring the level of LPC in a first biological sample obtained from the subject at a first time point prior to start of a treatment; (b) administering to the subject a treatment; (c) measuring the level of LPC in a second biological sample obtained from the subject at a second time point after start of the treatment, wherein the first and second biological samples are of the same type, and wherein the first and second time are in chronological order; (d) comparing the first measurement and the second measurement of LPC wherein the results of the comparison determines the course of LAM or the disease in the subject; (e) deciding a course of action for the subject based upon the results of the comparison in step (d) wherein the course of action is selected from the group consisting of terminating current treatment, modify current treatment, and maintained current treatment; and (f) executing the course of action.

In another embodiment, provided herein is a method of treating LAM, TSC-LAM and S-LAM, or a disease associated with a mutation in a TSC gene a subject, the method comprising: (a) measuring the level of LPC in a first biological sample obtained from the subject at a first time point prior to start of a treatment; (b) administering to the subject a treatment selected from the group consisting of hormone therapy, CK inhibition therapy, PLA2 inhibition therapy, mTORC1 inhibition therapy, and PI3Ks inhibition therapy; (c) measuring the level of LPC in a second biological sample obtained from the subject at a second time point after start of the treatment, wherein the first and second biological samples are of the same type, and wherein the first and second time are in chronological order; (d) comparing the first measurement and the second measurement of LPC wherein the results of the comparison determines the course of LAM or the disease in the subject; (e) deciding a course of action for the subject based upon the results of the comparison in step (d) wherein the course of action is selected from the group consisting of terminating current treatment, modify current treatment, and maintained current treatment; and (f) executing the course of action. In one embodiment of this treatment method, the method further comprises administering an additional or new treatment to the subject for treating the disease.

In one embodiment, provided herein is a method of assessing the efficacy of a drug in a subject, the method comprising: (a) measuring the level of LPC in a first biological sample obtained from the subject at a first time point, wherein the subject has LAM, LAM, TSC-LAM and S-LAM, or a disease associated with a mutation in a TSC gene; (b) measuring the level of LPC in a second biological sample obtained from the subject at a second time point, wherein the first and second biological samples are of the same type, and wherein the first and second time are in chronological order; (c) comparing the first measurement and the second measurement of LPC wherein the results of the comparison determines whether the drug is effective in the subject; and (d) deciding a course of action for the subject based upon the results of the comparison in step (c) wherein the course of action is selected from the group consisting of maintained current drug application, terminating current drug application, administer a new treatment and modify current drug application.

In one embodiment, provided herein is a method of assessing the efficacy of a drug in a subject, the method comprising: (a) administering a treatment to the subject, wherein the subject has LAM, LAM, TSC-LAM and S-LAM, or a disease associated with a mutation in a TSC gene; (b) measuring the level of LPC in a first biological sample obtained from the subject at a first time point after start of the treatment; (c) measuring the level of LPC in a second biological sample obtained from the subject at a second time point, wherein the first and second biological samples are of the same type, and wherein the first and second time are in chronological order; (d) comparing the first measurement and the second measurement of LPC wherein the results of the comparison determines whether the drug is effective in the subject; and (e) deciding a course of action for the subject based upon the results of the comparison in step (c) wherein the course of action is selected from the group consisting of maintained current drug application, terminating current drug application, administer a new treatment and modify current drug application.

In one embodiment, provided herein is a method of assessing the efficacy of a drug in a subject, the method comprising: (a) measuring the level of LPC in a first biological sample obtained from the subject at a first time point, wherein the subject has LAM, LAM, TSC-LAM and S-LAM, or a disease associated with a mutation in a TSC gene; (b) administering a treatment to the subject; (c) measuring the level of LPC in a second biological sample obtained from the subject at a second time point after the start of the treatment, wherein the first and second biological samples are of the same type, and wherein the first and second time are in chronological order; (d) comparing the first measurement and the second measurement of LPC wherein the results of the comparison determines whether the drug is effective in the subject; and (e) deciding a course of action for the subject based upon the results of the comparison in step (c) wherein the course of action is selected from the group consisting of maintained current drug application, terminating current drug application, administer a new treatment and modify current drug application.

Since the LAM cells were shown to have an elevated choline uptake, isotope-labeled choline can be used to visualize the increase choline uptake in these diseases disclosed herein. It is contemplated that there will be overall increased isotope-labeled choline in a subject has LAM, LAM, TSC-LAM and S-LAM, or a disease associated with a mutation in a TSC gene. It is also contemplated that increased isotope-labeled choline would be detected at the sites of proliferations of LAM cells and cells bearing in a TSC gene. Therefore, increased isotope-labeled choline signals can be used as an indicator to assess the disease activity in situations where there is no treatments and also in situations where there is treatment implemented, monitoring for recurrence of the disease after remission, track drug or treatment efficacy during treatment, and also locate site of disease activity.

Accordingly, in one embodiment, provided herein is a method of monitoring the disease activity of LAM, TSC-LAM and S-LAM, or a disease associated with a mutation in a TSC gene a subject, the method comprising: (a) imaging the intensity and location of carbon-11-labeled choline, fluorine 18-labeled choline or derivatives thereof in the subject at a first time; (b) imaging the intensity and location of carbon-11-labeled choline, fluorine 18-labeled choline or derivatives thereof in the subject at a second time, wherein the first and second time are in chronological order; (c) comparing the first imaging and the second imaging, wherein the results of the comparison determines the course of LAM or the disease in the subject; and (d) deciding a course of action for the subject based upon the results of the comparison in step (c) wherein the course of action is selected from the group consisting of maintained no treatment, terminating current treatment, modify current treatment, and maintained current treatment.

In one embodiment, provided herein is a method of monitoring the disease activity of LAM, TSC-LAM and S-LAM, or a disease associated with a mutation in a TSC gene a subject, the method comprising: (a) administering to the subject a treatment; (b) imaging the intensity and location of carbon-11-labeled choline, fluorine 18-labeled choline or derivatives thereof in the subject at a first time; (c) imaging the intensity and location of carbon-11-labeled choline, fluorine 18-labeled choline or derivatives thereof in the subject at a second time, wherein the first and second time are in chronological order; (d) comparing the first imaging and the second imaging, wherein the results of the comparison determines the course of LAM or the disease in the subject; and (e) deciding a course of action for the subject based upon the results of the comparison in step (d) wherein the course of action is selected from the group consisting of terminating current treatment, modify current treatment, and maintained current treatment. In one of this treatment method, the treatment selected from the group consisting of hormone therapy, CK inhibition therapy, PLA2 inhibition therapy, mTORC1 inhibition therapy, and PI3Ks inhibition therapy.

In one embodiment, provided herein is a method of monitoring the disease activity of LAM, TSC-LAM and S-LAM, or a disease associated with a mutation in a TSC gene a subject, the method comprising: (a) imaging the intensity and location of carbon-11-labeled choline; (b) administering to the subject a treatment, fluorine 18-labeled choline or derivatives thereof in the subject at a first time; (c) imaging the intensity and location of carbon-11-labeled choline, fluorine 18-labeled choline or derivatives thereof in the subject at a second time, wherein the first and second time are in chronological order; (d) comparing the first imaging and the second imaging, wherein the results of the comparison determines the course of LAM or the disease in the subject; and (e) deciding a course of action for the subject based upon the results of the comparison in step (d) wherein the course of action is selected from the group consisting of terminating current treatment, modify current treatment, and maintained current treatment. In one of this treatment method, the treatment selected from the group consisting of hormone therapy, CK inhibition therapy, PLA2 inhibition therapy, mTORC1 inhibition therapy, and PI3Ks inhibition therapy.

In one embodiment, provided herein is a method of treating LAM, TSC-LAM and S-LAM, or a disease associated with a mutation in a TSC gene in a subject, the method comprising: (a) imaging the intensity and location of carbon-11-labeled choline, fluorine 18-labeled choline or derivatives thereof in the subject at a first time; (b) imaging the intensity and location of carbon-11-labeled choline, fluorine 18-labeled choline or derivatives thereof in the subject at a second time, wherein the first and second time are in chronological order; (c) comparing the first imaging and the second imaging, wherein the results of the comparison determines the course of LAM or the disease in the subject; (d) deciding a course of action for the subject based upon the results of the comparison in step (c) wherein the course of action is selected from the group consisting of maintained no treatment, terminating current treatment, modify current treatment, administer a new treatment and maintain current treatment; and (f) executing the course of action. In one embodiment of this treatment method, the method further comprises administering a treatment to the subject for treating the disease.

In one embodiment, provided herein is a method of treating LAM, TSC-LAM and S-LAM, or a disease associated with a mutation in a TSC gene in a subject, the method comprising: (a) administering to the subject a treatment; (b) imaging the intensity and location of carbon-11-labeled choline, fluorine 18-labeled choline or derivatives thereof in the subject at a first time; (c) imaging the intensity and location of carbon-11-labeled choline, fluorine 18-labeled choline or derivatives thereof in the subject at a second time, wherein the first and second time are in chronological order; (d) comparing the first imaging and the second imaging, wherein the results of the comparison determines the course of LAM or the disease in the subject; (e) deciding a course of action for the subject based upon the results of the comparison in step (d) wherein the course of action is selected from the group consisting of maintained no treatment, terminating current treatment, modify current treatment, administer a new treatment and maintain current treatment; and (f) executing the course of action. In one embodiment of this treatment method, the method further comprises administering a treatment to the subject for treating the disease. In one of this treatment method, the treatment selected from the group consisting of hormone therapy, CK inhibition therapy, PLA2 inhibition therapy, mTORC1 inhibition therapy, and PI3Ks inhibition therapy.

In one embodiment, provided herein is a method of treating LAM, TSC-LAM and S-LAM, or a disease associated with a mutation in a TSC gene in a subject, the method comprising: (a) imaging the intensity and location of carbon-11-labeled choline, fluorine 18-labeled choline or derivatives thereof in the subject at a first time; (b) administering to the subject a treatment; (c) imaging the intensity and location of carbon-11-labeled choline, fluorine 18-labeled choline or derivatives thereof in the subject at a second time, wherein the first and second time are in chronological order; (d) comparing the first imaging and the second imaging, wherein the results of the comparison determines the course of LAM or the disease in the subject; (e) deciding a course of action for the subject based upon the results of the comparison in step (d) wherein the course of action is selected from the group consisting of maintained no treatment, terminating current treatment, modify current treatment, administer a new treatment and maintain current treatment; and (f) executing the course of action. In one of this treatment method, the treatment selected from the group consisting of hormone therapy, CK inhibition therapy, PLA2 inhibition therapy, mTORC1 inhibition therapy, and PI3Ks inhibition therapy.

In one embodiment, provided herein is a method of monitoring the recurrence of LAM, LAM, TSC-LAM and S-LAM, or a disease associated with a mutation in a TSC gene in a subject, the method comprising: (a) imaging the intensity and location of carbon-11-labeled choline, fluorine 18-labeled choline or derivatives thereof in the subject at a first time point when the subject is in remission; (b) imaging the intensity and location of carbon-11-labeled choline, fluorine 18-labeled choline or derivatives thereof in the subject at a second time during remission, wherein the first and second time are in chronological order; (c) comparing the first imaging and the second imaging, wherein the comparison indicates recurrence or maintained remission of LAM in the subject.

In one embodiment, provided herein is a method of assessing the efficacy of a drug in a subject, the method comprising: (a) imaging the intensity and location of carbon-11-labeled choline, fluorine 18-labeled choline or derivatives thereof in the subject at a first time, wherein the subject has LAM, LAM, TSC-LAM and S-LAM, or a disease associated with a mutation in a TSC gene; (b) imaging the intensity and location of carbon-11-labeled choline, fluorine 18-labeled choline or derivatives thereof in the subject at a second time, wherein the first and second time are in chronological order; (c) comparing the first imaging and the second imaging, wherein the results of the comparison determines whether the drug is effective in the subject; and (d) deciding a course of action for the subject based upon the results of the comparison in step (c) wherein the course of action is selected from the group consisting of maintain no treatment, maintained current drug application, terminating current drug application, administer a new treatment and modify current drug application. In one of this method, the treatment selected from the group consisting of hormone therapy, CK inhibition therapy, PLA2 inhibition therapy, mTORC1 inhibition therapy, and PI3Ks inhibition therapy.

In one embodiment, provided herein is a method of assessing the efficacy of a drug in a subject, the method comprising: (a) administering a treatment to the subject, wherein the subject has LAM, LAM, TSC-LAM and S-LAM, or a disease associated with a mutation in a TSC gene; (b) imaging the intensity and location of carbon-11-labeled choline, fluorine 18-labeled choline or derivatives thereof in the subject at a first time; (c) imaging the intensity and location of carbon-11-labeled choline, fluorine 18-labeled choline or derivatives thereof in the subject at a second time, wherein the first and second time are in chronological order, wherein the first and second time are in chronological order; (d) comparing the first imaging and the second imaging, wherein the results of the comparison determines whether the drug is effective in the subject; and (e) deciding a course of action for the subject based upon the results of the comparison in step (d) wherein the course of action is selected from the group consisting of maintained current drug application, terminating current drug application, administer a new treatment and modify current drug application. In one of this method, the treatment selected from the group consisting of hormone therapy, CK inhibition therapy, PLA2 inhibition therapy, mTORC1 inhibition therapy, and PI3Ks inhibition therapy.

In one embodiment, provided herein is a method of assessing the efficacy of a drug in a subject, the method comprising: (a) imaging the intensity and location of carbon-11-labeled choline, fluorine 18-labeled choline or derivatives thereof in the subject at a first time, wherein the subject has LAM, LAM, TSC-LAM and S-LAM, or a disease associated with a mutation in a TSC gene; (b) administering a treatment to the subject; (c) imaging the intensity and location of carbon-11-labeled choline, fluorine 18-labeled choline or derivatives thereof in the subject at a second time, wherein the first and second time are in chronological order, wherein the first and second time are in chronological order; (d) comparing the first imaging and the second imaging, wherein the results of the comparison determines whether the drug is effective in the subject; and (e) deciding a course of action for the subject based upon the results of the comparison in step (d) wherein the course of action is selected from the group consisting of maintained current drug application, terminating current drug application, administer a new treatment and modify current drug application. In one of this treatment method, the treatment selected from the group consisting of hormone therapy, CK inhibition therapy, PLA2 inhibition therapy, mTORC1 inhibition therapy, and PI3Ks inhibition therapy.

In one embodiment of any assessing method described herein, the method the method further comprises administering a treatment to the subject for treating the disease, wherein the disease is LAM, LAM, TSC-LAM and S-LAM, or a disease associated with a mutation in a TSC gene.

In one embodiment of any assessing method described herein, the treatment is administered in a time period selected from the group consisting of before the first measurement of LPC or imaging at the first time point; after the first measurement of LPC or imaging at the first time point but before the second measurement of LPC or imaging at the second time point; after the second measurement of LPC or imaging at the second time point, and after deciding a course of action for the subject.

In one embodiment of any treatment method described herein, the method further comprises administering a treatment to the subject in need. In some embodiments, the treatment is a new, modified or an additional treatment, in addition to current treatment.

In one embodiment of any monitoring or assessing method described herein, the method further comprises administering a treatment to the subject for treating the disease.

In one embodiment of any monitoring or assessing method described herein, the treatment is administered in a time period selected from the group consisting of before the first measurement of LPC or imaging at the first time point; after the first measurement of LPC or imaging at the first time point but before the second measurement of LPC or imaging at the second time point; after the second measurement of LPC or imaging at the second time point, and after deciding a course of action for the subject.

In one embodiment, provided herein is a method of treating LAM, TSC-LAM and S-LAM, or a disease associated with a mutation in a TSC gene in a subject comprising administering a therapeutically effective amount of a CK inhibitor and/or a therapeutically effective amount of a PLA2 inhibitor to the subject in need thereof. In one embodiment of this method, an elevated level of LPC in a biological sample from the subject is reduced and this reduced LPC level is closer by at least 5% to a predetermined reference LPC level. In one embodiment of this method, an elevated level of LPC in a biological sample from the subject is reduced by at least 5% from an original elevated level of LPC determined prior to administering the therapeutically effective amount of a CK inhibitor and/or a therapeutically effective amount of a PLA2 inhibitor.

In one embodiment, provided herein is a method of treating LAM, TSC-LAM and S-LAM, or a disease associated with a mutation in a TSC gene in a human subject comprising (a) diagnosing a subject as having LAM, TSC-LAM and S-LAM, or a disease associated with a mutation in a TSC gene, or diagnosing a subject as having a recurrence of LAM, TSC-LAM and S-LAM, or a disease associated with a mutation in a TSC gene by any method described herein comprising measuring the level of LPC in a biological sample obtained from the subject; and (b) administering a therapeutically effective amount of a CK inhibitor and/or a therapeutically effective amount of a PLA2 inhibitor to the subject in need thereof.

In one embodiment, provided herein is a method of treating LAM, TSC-LAM and S-LAM, or a disease associated with a mutation in a TSC gene in a human subject comprising (a) diagnosing a subject as having LAM, TSC-LAM and S-LAM, or a disease associated with a mutation in a TSC gene, or diagnosing a subject as having a recurrence of LAM, TSC-LAM and S-LAM, or a disease associated with a mutation in a TSC gene by any method described herein comprising imaging the intensity and location of carbon-11-labeled choline, fluorine 18-labeled choline or derivatives in the subject; and (b) administering a therapeutically effective amount of a CK inhibitor and/or a therapeutically effective amount of a PLA2 inhibitor to the subject in need thereof.

In one embodiment of any methods described herein, the level of LPC in a biological sample obtained from the subject is elevated when compared to the LPC level of a healthy subject or a predetermined LPC level of healthy subjects. In one embodiment of any methods described herein, an elevated LPC level indicates the likelihood of LAM, TSC-LAM and S-LAM, or a disease associated with a mutation in a TSC gene in the subject. In one embodiment of any methods described herein, an elevated LPC level is diagnostic of LAM, TSC-LAM and S-LAM, or a disease associated with a mutation in a TSC gene in the subject.

In one embodiment, by “elevated” with respect to the LPC levels in a biological sample obtained from a test subject means that the LPC level is at least more than 5% higher, greater or increased over the LPC level of a healthy subject who does not have LAM, TSC-LAM and S-LAM, or a disease associated with a mutation in a TSC gene. In other embodiments, an “elevated” LPC level is at least 10%, at least 15%, at least 20%, at least 25%, at least 30%, or more higher, greater or increased over the LPC level of a healthy subject who does not have LAM, TSC-LAM and S-LAM, or a disease associated with a mutation in a TSC gene.

In another embodiment, by “elevated” with respect to the LPC levels in a biological sample obtained from a test subject means that the LPC level is at least more than 5% higher, greater or increased over a predetermined reference LPC level of healthy subjects who do not have LAM, TSC-LAM and S-LAM, or a disease associated with a mutation in a TSC gene. In other embodiments, an “elevated” LPC level is at least 10%, at least 15%, at least 20%, at least 25%, at least 30%, or more higher, greater or increased over a predetermined LPC level of healthy subjects who do not have LAM, TSC-LAM and S-LAM, or a disease associated with a mutation in a TSC gene.

In one embodiment of any methods described herein, the species of LPC analyzed are selected from the selected from the group consisting of: C14:0 LPC; C16:0 LPC; C16:1 LPC; C18:0 LPC; C18.1 LPC; (1) C18:3 LPC; C18:2 LPC; C20:3 LPC; C20:4 LPC; and C22:6 LPC.

In one embodiment of any methods described herein, the LPC analyzed for comparison purposes are the same species in the test subject and the healthy subject. This means that the C14:0 LPC level of the test subject is compared with the C14:0 LPC level of the healthy subject; the C16:0 LPC level of the test subject is compared with the C16:0 LPC level of the healthy subject; the C18:3 LPC level of the test subject is compared with the C18:3 LPC level of the healthy subject; the C20:3 LPC level of the test subject is compared with the C20:3 LPC level of the healthy subject; the C22:6 LPC level of the test subject is compared with the C22:6 LPC level of the healthy subject and so forth.

In one embodiment of any methods described herein, at least one LPC is measured. In another embodiment of any methods described herein, more than one LPC is measured. In other embodiments of any methods described herein, at least two, at least three, at least four, at least five, at least six, at least seven, at least eight, at least nine, at least ten or more LPC are measured. In other embodiments of any methods described herein, two, three, four, five, six, seven, eight, nine, ten or more LPC are measured.

In another embodiment of any methods described herein, C16:0, C18:0, C18:1 and C20:4 LPC are measured. In another embodiment of any methods described herein, C16:0, C18:0 and C20:4 LPC are measured. In another embodiment of any methods described herein, C18:3 and C20:4 LPC are measured. In another embodiment of any methods described herein, C16:0, C18:0, C18:3 and C20:4 LPC are measured. In another embodiment of any methods described herein, C16:1, C18:0, C18:1 and C20:4 LPC are measured. In another embodiment of any methods described herein, C16:1, C18:3, C18:1 and C20:4 LPC are measured. In another embodiment of any methods described herein, C16:1 and C18:3 LPC are measured. In another embodiment of any methods described herein, C16:1, C18:3 and C20:4 LPC are measured. In another embodiment of any methods described herein, C16:0 and C20:4 LPC are measured. In another embodiment of any methods described herein, C16:1 and C20:4 LPC are measured. In another embodiment of any methods described herein, C18:1 and C20:4 LPC are measured. In another embodiment of any methods described herein, C16:0 and C18:0 LPC are measured. In another embodiment of any methods described herein, C16:0, C18:0, C18:1 and C20:4 LPC are measured. In another embodiment of any methods described herein, C16:0, C18:0, C18:1 and C18:3 LPC are measured.

In one embodiment of any method described herein, the LPC is measured by liquid chromatography coupled to mass spectrometry (LC-MS), enzymatic measurements, or gas chromatography coupled to chemical ionization mass spectrometry (GC-CIMS). In another embodiment of any method described herein, the LPC is measured or analyzed by any method known in the art. For example, see Kishimoto T. (2002) Clin Biochem. 35:411-6; and Zhao Z. (2007) J. Clin. Onco. 25:2696-2701.

In one embodiment of any method described herein, the analytical methods of measuring LPC levels in the test subject and the healthy subject are the same. For example, LC-MS are used for measuring both the LPC levels in the test subject and the healthy subject.

In one embodiment of any method described herein, the treatment applied to the subject results in the level of LPC in a biological sample from the treated subject being reduced or decreased from the original elevated level, wherein the reduction is at least 5% compared to the original elevated level. In other embodiments, the reduction in the LPC level after treatment is at least 5%, at least 10%, at least 15%, at least 20%, at least 25%, at least 30%, or more higher compared to the original elevated level.

In another embodiment of any method described herein, the treatment applied to the subject results in the level of LPC in a biological sample from the treated subject being less than 5% “elevated” compared the LPC level of a healthy subject or a predetermined reference LPC level of healthy subjects. In other embodiments, the treatment applied to the subject results in the level of LPC becoming less than 4.5%, less than 4.0%, less than 3.5%, less than 3.0%, less than 2.5%, less than 2.0%, less than 1.5%, less than 1.0%, or less than 0.5%, compared the LPC level of a healthy subject or a predetermined reference LPC level of healthy subjects, including all the possible percentages between 5% to 0% to the second decimal place.

In one embodiment of any method described herein, other additional treatments described herein are contemplated.

In one embodiment of any method described herein, the method described herein can be performed when the subject has had no treatment, ie., without treatment. In another of any method described herein, the method described herein can be performed when the subject is being treated, ie., with treatment. In another embodiment of any method described herein, the subject had previously been treated and is now in remission and is currently not on any treatment for the disease.

As used herein, the term “remission” with reference to LAM, TSC-LAM and S-LAM, or a disease associated with a mutation in a TSC gene refers to the state of absence of disease activity in patients with the possibility of return of disease activity. In one embodiment, “remission” refers to the situation where a subject's LPC level is less than 5% higher compare to the LPC level of a healthy subject or the predetermined reference LPC level of healthy subjects. In one embodiment, “remission” refers to the situation where the subject has no detectable cysts by known methods such as imaging. In one embodiment, a subject is in “remission” when the subject has recovered and has at least 80% of pulmonary function prior to the disease.

In one embodiment of any method described herein, the first time point is selected for the group consisting of prior to an application of a treatment, during the course of a first treatment, during the course of a second treatment, and the course of a subsequent treatment.

In one embodiment of any method described herein, the first time point is prior to an application of a treatment and this measurement is elevated compared to the LPC level of a healthy subject or a predetermined reference LPC level of healthy subjects. In another embodiment of any method described herein, the first time point is during the course of a treatment, either first, second or subsequent treatment, and this first measurement is elevated compared to the LPC level of a healthy subject or a predetermined reference LPC level of healthy subjects.

In one embodiment of any method described herein, the first time point is prior to an application of a treatment and this measurement is not elevated compared to the LPC level of a healthy subject or a predetermined reference LPC level of healthy subjects, i.e., the first LPC level is less than 5% higher compared to the LPC level of a healthy subject or a predetermined reference LPC level of healthy subjects. In another embodiment of any method described herein, the first time point is during the course of a treatment, either first, second or subsequent treatment, and this first measurement is not elevated compared to the LPC level of a healthy subject or a predetermined reference LPC level of healthy subjects.

In one embodiment of any method described herein, the second time point is selected for the group consisting of prior to an application of a treatment, during the course of a first treatment, during the course of a second treatment, and the course of a subsequent treatment. In one embodiment of any method described herein, the second time point is prior to an application of a treatment and this measurement is elevated compared to the LPC level of a healthy subject or a predetermined reference LPC level of healthy subjects. In another embodiment of any method described herein, the second time point is during the course of a treatment, either first, second or subsequent treatment, and this first measurement is elevated compared to the LPC level of a healthy subject or a predetermined reference LPC level of healthy subjects.

In one embodiment of any method described herein, the second time point is selected for the group consisting of prior to an application of a treatment, during the course of a first treatment, during the course of a second treatment, and the course of a subsequent treatment. In one embodiment of any method described herein, the second time point is prior to an application of a treatment and this measurement is not elevated compared to the LPC level of a healthy subject or a predetermined reference LPC level of healthy subjects. That is the second LPC level is less than 5% higher compared to the LPC level of a healthy subject or a predetermined reference LPC level of healthy subjects. In another embodiment of any method described herein, the second time point is during the course of a treatment, either first, second or subsequent treatment, and this first measurement is elevated compared to the LPC level of a healthy subject or a predetermined reference LPC level of healthy subjects.

In one embodiment of any method described herein, the course of LAM or a disease associated with a mutation in a TSC gene in the subject is selected from the group consisting of no further progression, continued progression and regression.

In one embodiment of any method described herein, one is comparing the LPC levels measured at two time points, a first and a second measurement during which the subject is not being treated for LAM, or not being treated for TSC-LAM, or not being treated for S-LAM, or not being treated for a disease associated with a mutation in a TSC gene. The comparison allows the physician to determine whether LAM or the disease condition is progressing or holding status quo with no further progressing, and to make a decision whether a medical therapeutic intervention is warranted when the LAM or the disease condition is progressing in the subject.

Accordingly, in one embodiment of any method described herein, both the first and second measurements of LPC are prior to an application of a treatment, and wherein the second measurement of LPC is the same as or is no more than 5% over the first measurement of LPC indicates that there is no further progression of LAM or the disease condition in the subject, and the course of action is to maintain no treatment in the subject. In this embodiment, the physician can periodically survey for the progression of LAM or the disease condition in the subject, and when there is an indication that LAM or the disease condition is progressing, then the physician decide to initiate a treatment plan in the subject.

In another embodiment of any method described herein, both the first and second measurements of LPC are prior to an application of a treatment, and wherein the second measurement of LPC is at least 5% over or higher compared to the first measurement of LPC indicates that there is progression of LAM or the disease condition in the subject, and the course of action is to initiate a treatment in the subject. In other embodiments, the second measurement of LPC is at least 10%, at least 20%, at least 30%, at least 40%, at least 50% over or higher than the first measurement of LPC.

In one embodiment of any method described herein, one is comparing the LPC levels at two time points, a first and a second during which the subject is being treated for LAM, TSC-LAM and S-LAM, or a disease associated with a mutation in a TSC gene. The comparison allows the physician to determine whether the treatment is effective in regressing the disease condition or whether the treatment is merely stopping the disease condition from any further progressing, or whether the treatment is totally ineffective in halting the progression of the disease condition.

In addition, the method allows the physician to continually monitor the efficacy of a treatment that had been effective previously. This is because some times, LAM, TSC-LAM and S-LAM, or a disease associated with a mutation in a TSC gene can develop resistance to a specific treatment, e.g., to a specific drug etc.

In one embodiment of any method described herein, both the first and second measurements of LPC are during the course of a first or second or subsequent treatment, wherein the first and second measurements are during the same treatment, wherein the second measurement of LPC during the course of the treatment is at least lower by 5% compared to the first measurement of LPC indicates regression of LAM or the disease condition in the subject, and that the current treatment is effective and is to be maintained. In other embodiments, the second measurement of LPC is at least lower by 4.5%, at least lower by 4.0%, at least lower by 3.5%, at least lower by 3.0%, at least lower by 2.5%, at least lower by 2.0%, at least lower by 1.5%, at least lower by 1.0%, or at least lower by 0.5% compared to the first measurement of LPC. In one embodiment of any method described herein, both elevated at least 5% higher than a predetermined reference LPC level.

In one embodiment of any method described herein, both the first and second measurements of LPC are during the course of a first or second or subsequent treatment, wherein the first and second measurements are during the same treatment, wherein the second measurement of LPC is the same or no more than 5% over the first measurement of LPC indicates no further progression of LAM or the disease in the subject, and that the current treatment is effective in preventing further progression of the disease and that the treatment is to be maintained or modified to promote regression. In one embodiment of any method described herein, both elevated at least 5% higher than a predetermined reference LPC level.

In one embodiment of any method described herein, both the first and second measurements of LPC are during the course of a first or second or subsequent treatment, wherein the first and second measurements are during the same treatment, wherein the second measurement of LPC is during the course of the treatment remains the same as or is higher by at least 5% over the first measurement of LPC indicates continued progression of LAM or the disease condition in the subject, and that the current treatment is ineffective and is to be terminated or modified. In one embodiment, the first LPC measurement is elevated and is indicative of LAM, TSC-LAM and S-LAM, or a disease associated with a mutation in a TSC gene in the subject. In other embodiments, the second measurement of LPC is at least 10%, at least 20%, at least 30%, at least 40%, at least 50% higher than the first measurement of LPC. In one embodiment of any method described herein, a new treatment or an additional treatment is implemented in view of the lack of efficacy of the prior treatment. In one embodiment of any method described herein, both elevated at least 5% higher than a predetermined reference LPC level.

In one embodiment of any method described herein, one is comparing the LPC levels obtained prior to an application of a treatment with the LPC levels obtained during a treatment in a subject. The comparison allows the physician to determine whether the treatment is effective in regressing the disease condition or whether the treatment is merely stopping the disease condition from any further progressing, or whether the treatment is totally ineffective in halting the progression of the disease condition.

In one embodiment of any method described herein, the first measurement of LPC is prior to an application of a treatment and the second measurement of LPC is during the course of a first or second or subsequent treatment, wherein the second measurement of LPC during the course of the treatment is the same as or elevated at least 5% over or higher than the first measurement of LPC prior to start of the treatment indicates continued progression of LAM or the disease condition in the subject, and that the current treatment is ineffective and is to be terminated or modified. In one embodiment, the first LPC measurement is elevated and is indicative of LAM, TSC-LAM and S-LAM, or a disease associated with a mutation in a TSC gene in the subject. In other embodiments, the second measurement of LPC is at least 10%, at least 20%, at least 30%, at least 40%, at least 50% higher than the first measurement of LPC.

In one embodiment of any method described herein, the first measurement of LPC is prior to an application of a treatment and the second measurement of LPC is during the course of the first or second or subsequent treatment, wherein the second measurement of LPC is at least lower by 5% compared to the first measurement of LPC indicates regression of LAM or the disease condition in the subject, and that the current treatment is effective and is to be maintained. In other embodiments, the second measurement of LPC is at least lower by 4.5%, at least lower by 4.0%, at least lower by 3.5%, at least lower by 3.0%, at least lower by 2.5%, at least lower by 2.0%, at least lower by 1.5%, at least lower by 1.0%, or at least lower by 0.5% compared to the first measurement of LPC.

In one embodiment of any method described herein, one is comparing the LPC level during a first treatment with the LPC level during a second treatment. All the treatments are on the same subject. The comparison allows the physician to compare the effectiveness of the two treatments in the subject. For example, the first treatment was evaluated to be ineffective and was terminated. Then a second treatment is administered to the subject. If the LPC level during a second treatment is lower than that of the first treatment, this would indicate that the second treatment is effective compared to the first treatment. Conversely, if the LPC level during a second treatment is the same or higher than that of the first treatment, this would indicate that the second treatment is not any more effective than the first treatment.

In one embodiment of any method described herein, the first measurement of LPC is a first treatment and the second measurement of LPC is during the course of the second or subsequent treatment, wherein the second measurement of LPC is the same as or elevated at least 5% over or higher that the first measurement of LPC indicates continued progression of LAM or the disease condition in the subject, and that the second or subsequent treatment is ineffective and is to be terminated or modified. In other embodiments, the second measurement of LPC is at least 10%, at least 20%, at least 30%, at least 40%, at least 50% higher than the first measurement of LPC.

In one embodiment of any method described herein, the first measurement of LPC is a first treatment and the second measurement of LPC is during the course of the second or subsequent treatment, wherein the second measurement of LPC is at least lower by 5% compared to the first measurement of LPC indicates regression of LAM or the disease condition in the subject, and that the second or subsequent treatment is effective and is to be maintained. In other embodiments, the second measurement of LPC is at least lower by 4.5%, at least lower by 4.0%, at least lower by 3.5%, at least lower by 3.0%, at least lower by 2.5%, at least lower by 2.0%, at least lower by 1.5%, at least lower by 1.0%, or at least lower by 0.5% compared to the first measurement of LPC.

As used herein, the term “progressing” or “progression” when used in the context of LAM, TSC-LAM and S-LAM, or a disease associated with a mutation in a TSC gene refers to the disease condition continuing along the natural course of the disease. For example, the disease condition is characterized by continued proliferation of smooth-muscle like cells, continued growth of associated tumors, continued production of cysts, pulmonary function and exercise function etc., in the subject.

As used herein, the term “disease condition” or “disease” refers to LAM, TSC-LAM and S-LAM, or a disease associated with a mutation in a TSC gene.

In some embodiments, the phrase “a disease associated with a mutation in a TSC gene” refers to LAM, TSC-LAM and S-LAM.

As used herein, the term “regressing’ or “regression” when used in the context of LAM, TSC-LAM and S-LAM, or a disease associated with a mutation in a TSC gene refers to the disease condition taking a turn opposite from the natural course of the disease. For example, there is reduced proliferation of smooth-muscle like cells, less or slower production of cysts, shrinkage of associated tumors, improved pulmonary function and exercise function etc., in the subject.

As used herein, the term “holding status quo” when used in the context of LAM, TSC-LAM and S-LAM, or a disease associated with a mutation in a TSC gene refers to when there is no change in the disease condition, there is neither progression nor regression of the disease condition. For example, there is no increased or reduced proliferation of smooth-muscle like cells, no increased or reduced production of cysts, no shrinkage or further growth of associated tumors, and no improved or improved pulmonary function and exercise function in the subject.

It is contemplated that during the course of LAM or the disease condition in the subject when the subject is not being treated, there will be several measurements of LPC levels made. It is contemplated that a later made measurement of LPC will be compared with an earlier made measurement of LPC in order to evaluate whether the course of LAM or the disease condition is progressing, holding status quo, or regressing. A later made measurement of LPC is a measurement made after an earlier made measurement of LPC with respect to chronological time.

It is also contemplated that during the course of a treatment of LAM or the disease condition in the subject, there will be several measurements of LPC levels made. It is contemplated that a later made measurement of LPC will be compared with an earlier made measurement of LPC in order to evaluate whether the course of LAM or the disease condition is progressing, holding status quo, or regressing. A later made measurement of LPC is a measurement made after an earlier made measurement of LPC with respect to chronological time.

In one embodiment of any method described herein, the predetermined reference LPC level comprises the average LPC level measurements from a plurality of healthy human subjects without LAM.

In one embodiment of any method described herein, the predetermined reference LPC level comprises the average LPC level measurements from a plurality of healthy human subjects without TSC-LAM.

In one embodiment of any method described herein, the predetermined reference LPC level comprises the average LPC level measurements from a plurality of healthy human subjects without S-LAM.

In one embodiment of any method described herein, the predetermined reference LPC level comprises the average LPC level measurements from a plurality of healthy human subjects without a disease associated with a mutation in a TSC gene.

In one embodiment of any method described herein, the healthy subject has not exhibited any common respiratory ailment that is known.

In one embodiment of any method described herein, a plurality of healthy human subjects is at least five healthy human subjects without LAM, or without TSC-LAM, or without S-LAM, or without a disease associated with a mutation in a TSC gene. In other embodiments of any method described herein, a plurality of healthy human subjects is at least ten, 15, 20, 25, 30, 35 or more healthy human subjects without LAM, or without TSC-LAM, or without S-LAM, or without a disease associated with a mutation in a TSC gene.

In one embodiment of any method described herein, the plurality of healthy human subjects comprised of subjects who do not have cancer, ie., diagnosed with cancer.

In one embodiment of any method described herein, the plurality of healthy human subjects is comprised of female subjects.

In one embodiment of any method described herein, the plurality of healthy human subjects is comprised of female subjects of the same age range. For example, female subjects in their 20s, in their 30s, in their 40s, in their 50s or in their 60s.

In one embodiment of any method described herein, the plurality of healthy human subjects is comprised of female subjects of the reproductive age.

In one embodiment of any method described herein, the plurality of healthy human subjects is comprised of female subjects of the reproductive age between 12-55, 12-60, 15-50 years old. In other embodiments of any method described herein, the healthy human subjects are between the ages of 15-20, 15-25, 15-30, 15-35, 15-40, 15-45, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, and 45-50 years old.

In one embodiment of any method described herein, the LPC level measurements from a plurality of healthy human subjects, ie., the predetermined LPC level, and the LPC level measurements from the test subject are measured by the same method. For example, if the predetermined LPC level is measured by LC-MS, then the LPC level measurements from the test subject is also measured by LC-MS; if the predetermined LPC level is measured by thin-layer chromatography, then the LPC level measurements from the test subject is also measured by thin-layer chromatography; and if the predetermined LPC level is measured by enzymatic analysis, then the LPC level measurements from the test subject is also measured by enzymatic analysis.

In one embodiment of any method described herein, both first measurement and the second measurement of LPC are elevated above the predetermined reference LPC level indicates continual LAM or the disease in the subject. In one embodiment of any method described herein, both first measurement and the second measurement of LPC are elevated at least 5% above the predetermined reference LPC level indicates continual LAM or the disease in the subject. In other embodiments of any method described herein, both first measurement and the second measurement of LPC are elevated at least 10%, at least 15%, at least 20%, at least 25%, at least 30%, or more above the predetermined reference LPC level indicates continual LAM or the disease in the subject.

In one embodiment of any method described herein, both first measurement and the second measurement of LPC are elevated above the predetermined reference LPC level, and the second measurement of LPC during the course of the treatment remains elevated but is reduced to a level that is at least 5% closer to the predetermined reference LPC level compared to first measurement indicates regression of LAM or the disease in the subject, and that the current treatment is effective and is to be maintained. In one embodiment, if there is no treatment administered, then the no treatment status is to be maintained.

In one embodiment of any method described herein, both first measurement and the second measurement of LPC are elevated above the predetermined reference LPC level, and the second measurement of LPC during the course of the treatment is the same or is at least 5% elevated compared to first measurement indicates continued progression of LAM or the disease condition in the subject, and that the current treatment is ineffective and is to be terminated or modified. In one embodiment, a new treatment or an additional treatment is implemented in view of the progression of the -treated LAM or disease and the prior treatment being ineffective. In one embodiment, if there is no treatment administered, then the physician can initiate a treatment plan in view of the progression of the non-treated LAM or disease. In other embodiments, the second measurement of LPC is at least 10%, at least 20%, at least 30%, at least 40%, at least 50% higher than the first measurement of LPC.

In one embodiment of any method described herein, the first imaging time point is selected for the group consisting of prior to an application of a treatment, during the course of a first treatment, during the course of a second treatment, and the course of a subsequent treatment.

In one embodiment of any method described herein, the first imaging time point is prior to an application of a treatment and this measurement, ie., the signal intensity and location signal, are elevated compared to the signal intensity and location signal levels of a healthy subject or a predetermined reference the signal intensity and location signal levels derived from healthy subjects. In another embodiment of any method described herein, the first imaging time point is during the course of a treatment, either first, second or subsequent treatment, and this first imaging signals, i.e., the signal intensity and location signal, are elevated compared to the signal intensity and location signal levels of a healthy subject or a predetermined reference the signal intensity and location signal levels of healthy subjects.

In one embodiment of any method described herein, the first imaging time point is prior to an application of a treatment and the signal intensity and location signal obtained are not elevated compared to the signal intensity and location signal levels of a healthy subject or a predetermined reference the signal intensity and location signal levels of healthy subjects, i.e., the first imaging data (the signal intensity and location signal) are less than 5% higher compared to the signal intensity and location signal levels of a healthy subject or a predetermined reference signal intensity and location signal levels of healthy subjects. In another embodiment of any method described herein, the first imaging time point is during the course of a treatment, either first, second or subsequent treatment, and this first measurement is not elevated compared to the signal intensity and location signal levels of a healthy subject or a predetermined reference signal intensity and location signal levels of healthy subjects.

In one embodiment of any method described herein, the second imaging time point is selected for the group consisting of prior to an application of a treatment, during the course of a first treatment, during the course of a second treatment, and the course of a subsequent treatment. In one embodiment of any method described herein, the second imaging time point is prior to an application of a treatment, and the signal intensity and location signal are both elevated compared to the signal intensity and location signal levels of a healthy subject or a predetermined reference signal intensity and location signal levels of healthy subjects. In another embodiment of any method described herein, the second time point is during the course of a treatment, either first, second or subsequent treatment, and the signal intensity and location signal are both elevated compared to the signal intensity and location signal levels of a healthy subject or a predetermined reference signal intensity and location signal levels of healthy subjects.

In one embodiment of any method described herein, the second imaging time point is selected for the group consisting of prior to an application of a treatment, during the course of a first treatment, during the course of a second treatment, and the course of a subsequent treatment. In one embodiment of any method described herein, the second imaging time point is prior to an application of a treatment and this imaging measurements (the signal intensity and location signal) is not elevated compared to the signal intensity and location signal levels of a healthy subject or a predetermined reference the signal intensity and location signal levels of healthy subjects. That is the data obtained from this second imaging is less than 5% higher compared to the signal intensity and location signal levels of a healthy subject or a predetermined reference signal intensity and location signal levels of healthy subjects. In another embodiment of any method described herein, the second time point is during the course of a treatment, either first, second or subsequent treatment, and the data obtained in this second imaging measurement are elevated compared to the signal intensity and location signal levels of a healthy subject or a predetermined reference the signal intensity and location signal levels of healthy subjects.

In one embodiment of any method described herein, one is comparing the isotope-labeled choline imaging signals measured at two time points, a first and a second measurement during which the subject is not being treated for LAM, or not being treated for TSC-LAM, or not being treated for S-LAM, or not being treated for a disease associated with a mutation in a TSC gene. The comparison allows the physician to determine whether LAM or the disease condition is progressing or holding status quo with no further progressing, and to make a decision whether a medical therapeutic intervention is warranted when the LAM or the disease condition is progressing in the subject.

Accordingly, in one embodiment of any method described herein, both the first and second measurements of isotope-labeled choline imaging signals are prior to an application of a treatment, and wherein the second imaging signals are the same as or is no more than 5% over the first imaging signals indicate that there is no further progression of LAM or the disease condition in the subject, and the course of action is to maintain no treatment in the subject. In this embodiment, the physician can periodically survey for the progression of LAM or the disease condition in the subject, and when there is an indication that LAM or the disease condition is progressing, then the physician decide to initiate a treatment plan in the subject.

In another embodiment of any method described herein, both the first and second measurements of the isotope-labeled choline imaging signals are prior to an application of a treatment, and wherein the second imaging signals are at least 5% over or higher compared to the first imaging signals indicate that there is progression of LAM or the disease condition in the subject, and the course of action is to initiate a treatment in the subject. In other embodiments, the second isotope-labeled choline imaging signals are at least 10%, at least 20%, at least 30%, at least 40%, at least 50% over or higher than the first isotope-labeled choline imaging signals.

In one embodiment of any method described herein, one is comparing the isotope-labeled choline imaging signals at two time points, a first and a second imaging signals are obtained during a period at which the subject is being treated for LAM, TSC-LAM and S-LAM, or a disease associated with a mutation in a TSC gene. The comparison allows the physician to determine whether the treatment is effective in regressing the disease condition or whether the treatment is merely stopping the disease condition from any further progressing, or whether the treatment is totally ineffective in halting the progression of the disease condition.

In addition, the method allows the physician to continually monitor the efficacy of a treatment that had been effective previously. This is because some times, LAM, TSC-LAM and S-LAM, or a disease associated with a mutation in a TSC gene can develop resistance to a specific treatment, e.g., to a specific drug etc.

In one embodiment of any method described herein, both the first and second isotope-labeled choline imaging signals are during the course of a first or second or subsequent treatment, wherein the first and second imaging signals are during the same treatment, wherein the second imaging signals during the course of the treatment are at least lower by 5% compared to the first the isotope-labeled choline imaging signals indicate regression of LAM or the disease condition in the subject, and that the current treatment is effective and is to be maintained. In other embodiments, the second imaging signals are at least lower by 4.5%, at least lower by 4.0%, at least lower by 3.5%, at least lower by 3.0%, at least lower by 2.5%, at least lower by 2.0%, at least lower by 1.5%, at least lower by 1.0%, or at least lower by 0.5% compared to the first imaging signals. In one embodiment of any method described herein, both the first and second isotope-labeled choline imaging signals are elevated at least 5% higher than a predetermined reference isotope-labeled choline imaging signals from healthy subjects.

In one embodiment of any method described herein, both the first and second isotope-labeled choline imaging signals are during the course of a first or second or subsequent treatment, wherein the first and second measurements are during the same treatment, wherein the second imaging signals are the same or no more than 5% over or higher than the first isotope-labeled choline imaging signals indicate no further progression of LAM or the disease in the subject, and that the current treatment is effective in preventing further progression of the disease and that the treatment is to be maintained or modified to promote regression. In one embodiment of any method described herein, both the first and second isotope-labeled choline imaging signals are elevated at least 5% higher than a predetermined reference isotope-labeled choline imaging signals from healthy subjects.

In one embodiment of any method described herein, both the first and second isotope-labeled choline imaging signals are during the course of a first or second or subsequent treatment, wherein the first and second measurements are during the same treatment, wherein the second imaging signals are during the course of the treatment remains the same as or is higher by at least 5% over the first imaging signals indicate continued progression of LAM or the disease condition in the subject, and that the current treatment is ineffective and is to be terminated or modified. In one embodiment, the first imaging signals are elevated and are indicative of LAM, TSC-LAM and S-LAM, or a disease associated with a mutation in a TSC gene in the subject. In other embodiments, the second imaging signals are at least 10%, at least 20%, at least 30%, at least 40%, at least 50% higher than the first imaging signals. In one embodiment of any method described herein, a new treatment or an additional treatment is implemented in view of the lack of efficacy of the prior treatment. In one embodiment of any method described herein, both the first and second isotope-labeled choline imaging signals are elevated at least 5% higher than a predetermined reference isotope-labeled choline imaging signals from healthy subjects.

In one embodiment of any method described herein, one is comparing the isotope-labeled choline imaging signals obtained prior to an application of a treatment with the isotope-labeled choline imaging signals obtained during a treatment in a subject. The comparison allows the physician to determine whether the treatment is effective in regressing the disease condition or whether the treatment is merely stopping the disease condition from any further progressing, or whether the treatment is totally ineffective in halting the progression of the disease condition.

In one embodiment of any method described herein, the first isotope-labeled choline imaging signals are obtained prior to an application of a treatment and the second isotope-labeled choline imaging signals are obtained during the course of a first or second or subsequent treatment, wherein the second imaging signals during the course of the treatment are the same as or elevated at least 5% over or higher than the first imaging signals obtained prior to start of the treatment indicate continued progression of LAM or the disease condition in the subject, and that the current treatment is ineffective and is to be terminated or modified. In one embodiment, the first imaging signals are elevated and are indicative of LAM, TSC-LAM and S-LAM, or a disease associated with a mutation in a TSC gene in the subject. In other embodiments, the second imaging signals are at least 10%, at least 20%, at least 30%, at least 40%, at least 50% higher than the first imaging signals.

In one embodiment of any method described herein, the first isotope-labeled choline imaging signals are obtained prior to an application of a treatment and the second isotope-labeled choline imaging signals are obtained during the course of the first or second or subsequent treatment, wherein the second imaging signals are at least lower by 5% compared to the first imaging signals indicate regression of LAM or the disease condition in the subject, and that the current treatment is effective and is to be maintained. In other embodiments, the second imaging signals is at least lower by 4.5%, at least lower by 4.0%, at least lower by 3.5%, at least lower by 3.0%, at least lower by 2.5%, at least lower by 2.0%, at least lower by 1.5%, at least lower by 1.0%, or at least lower by 0.5% compared to the first imaging signals.

In one embodiment of any method described herein, one is comparing the isotope-labeled choline imaging signals obtained during a first treatment with the isotope-labeled choline imaging signals obtained during a second treatment. All the treatments are on the same subject. The comparison allows the physician to compare the effectiveness of the two treatments in the subject. For example, the first treatment was evaluated to be ineffective and was terminated. Then a second treatment is administered to the subject. If the imaging signals during a second treatment is lower than that of the first treatment, this would indicate that the second treatment is effective compared to the first treatment. Conversely, if the imaging signals during a second treatment are the same or higher than that of the first treatment, this would indicate that the second treatment is not any more effective than the first treatment.

In one embodiment of any method described herein, the first isotope-labeled choline imaging signals are obtained in a first treatment and the second isotope-labeled choline imaging signals are obtained during the course of the second or subsequent treatment, wherein the second imaging signals are the same as or elevated at least 5% over or higher than the first imaging signals indicate continued progression of LAM or the disease condition in the subject, and that the second or subsequent treatment is ineffective and is to be terminated or modified. In other embodiments, the second imaging signals are at least 10%, at least 20%, at least 30%, at least 40%, at least 50% higher than the first isotope-labeled choline imaging signals.

In one embodiment of any method described herein, the first isotope-labeled choline imaging signals are obtained in a first treatment and the second isotope-labeled choline imaging signals are obtained during the course of the second or subsequent treatment, wherein the second imaging signals are at least lower by 5% compared to the first imaging signals indicate regression of LAM or the disease condition in the subject, and that the second or subsequent treatment is effective and is to be maintained. In other embodiments, the second imaging signals are at least lower by 4.5%, at least lower by 4.0%, at least lower by 3.5%, at least lower by 3.0%, at least lower by 2.5%, at least lower by 2.0%, at least lower by 1.5%, at least lower by 1.0%, or at least lower by 0.5% compared to the first imaging signals.

In one embodiment of any method described herein, the isotope-labeled choline imaging signals comprise signal intensity and location signal.

The signal intensity is the electric-field strength of the electromagnetic wave emitted from the isotope-labeled choline transmitting a signal. With increased uptake of choline in the described disease condition, there will be increased local concentration of isotope-labeled choline leading to increased signal intensity.

The location signals are signals indicating the location in the imaged body of the subject where there is local concentration of isotope-labeled choline. These locations are collections of LAM cells or cells that have a mutation in a TSC gene. These cells have increased uptake of choline. Accordingly, increased signal intensity and/or increased location signals indicate progression of LAM, TSC-LAM and S-LAM, or a disease associated with a mutation in a TSC gene in the subject. Conversely if there is not further increased signal intensity and/or increased location signals in the subject, that means the LAM, TSC-LAM and S-LAM, or a disease associated with a mutation in a TSC gene is not progressing and is holding status quo in the subject.

In one embodiment of any method described herein, the second imaging has lower intensity and location signal by at least 5% compared to the first imaging indicates regression of LAM or the disease in the subject, and that the course of action is to be maintained.

In one embodiment of any method described herein, the second imaging intensity and location signals remain the same as or are higher by at least 5% over the first imaging indicates progression of LAM or the disease in the subject, and that the course of action is to be terminated or modified.

In one embodiment of any method described herein, the second imaging has lower intensity and location signal by at least higher than 5% compared to the first imaging indicates recurrence of LAM in the subject, and that treatment is needed.

In one embodiment of any method described herein, the second imaging intensity and location signals remain the same as or are no more than 5% over the first imaging indicates maintained remission of LAM in the subject, and no treatment is needed.

In one embodiment of any method described herein, the subject is being treated with a drug for treating LAM, LAM, TSC-LAM and S-LAM, or a disease associated with a mutation in a TSC gene; and wherein the subject is continual being treated with the drug.

In one embodiment of any method described herein, the isotope-labeled choline used in the imaging are 11C or 18F label choline or their derivatives.

In one embodiment of any method described herein, the subject is administered choline that is labeled with carbon-11 or fluorine-18. Carbon-11 and fluorine-18 are radioactive positron emitters which enable medical imaging of the carbon-11 or fluorine-18 labeled choline on a positron emission tomography (PET) scanner.

In one embodiment of any method described herein, the imaging is performed with a positron emission tomography (PET) scanner. In another embodiment of any method described herein, the imaging is performed with a PET/computed tomography scanner. A skilled physician or a skilled radiologist will be able to perform the imaging scans and perform comparison analysis of the images obtained at the various time points described or with the predetermined levels of signal intensity and location signals for a healthy subject or a plurality of healthy subjects.

Choline is a water-soluble essential nutrient. It is usually grouped within the B-complex vitamins. Choline generally refers to the various quaternary ammonium salts containing the N,N,N-trimethylethanolammonium cation.

The cation appears in the head groups of phosphatidylcholine and sphingomyelin, two classes of phospholipid that are abundant in cell membranes. Choline is the precursor molecule for the neurotransmitter acetylcholine which is involved in many functions including memory and muscle control.

Accordingly, choline and its metabolites are needed for three main physiological purposes: structural integrity and signaling roles for cell membranes, cholinergic neurotransmission (acetylcholine synthesis), and a major source for methyl groups via its metabolite, trimethylglycine (betaine) which participates in the S-adenosylmethionine (SAMe) synthesis pathways.

The U.S. food and drug administration (FDA) has recently approved the production and use of 11-C choline injection in conjunction with PET imaging to detect recurrent prostate cancer.

In one embodiment of any method described herein, the 11-C or 18-F choline or derivatives thereof are administered intravenously by injection is to produce an image that helps a skilled physician or radiologist to locate specific body sites of increased choline uptake as well as locations of new proliferating LAM cells or cells having a disease associated with a mutation in a TSC gene. After there is positive increased choline uptake and locations of such choline uptake, additional medical procedures can be taken, such as follow-up tissue sampling and testing in the subject for increased LPC and/or VEGF-D.

The recommended dose and administration are well known in the art, for example, as described in the FDA publication document 2012/203155s0001b1.pdf at the FDA website.

In one embodiment of any method described herein, the recommended dose of 11-C or 18-F choline or derivatives thereof is 370 to 740 MBq (10 to 20 mCi) to be administered as a bolus intravenous injection. The radioactivity dose (370 to 740 MBq, 10 to 20 mCi) is chosen based on patient body dimensions and the characteristics of the image acquisition system. It is recommended that image acquisition commence immediately after the injection.

There are two TSC genes in the human genome, TSC1 and TSC2 which codes for hamartin and tuberin respectively. Hamartin and tuberin function as a complex to interact with Rheb GTPase, thereby sequestering Rheb GTPase from activating mTOR signaling. In the absence of Rheb GTPase, there is deregulation of the mTOR signaling pathway such that the pathway is hyperactivei, resulting in an increased in mTOR signaling. The mammalian target of rapamycin (mTOR) signalling pathway is a major player controlling cell growth and cell division. The kinase, mTOR, is a master regulator of protein synthesis that couples nutrient sensing to cell growth. Defects in the mTOR signalling pathway can result in loss of control in cell growth and cell division, and subsequently a predisposition to forming tumors.

Accordingly, in one embodiment of any method described herein, the mutation is in the TSC1 gene and/or TSC 2 gene. In one embodiment of any method described herein, the mutation is in the TSC1 gene. In one embodiment of any method described herein, the mutation is in the TSC2 gene. In another embodiment of any method described herein, there mutations in both the TSC1 and TSC2 genes.

In one embodiment of any method described herein, the mutation results in no protein production or no functional protein.

In one embodiment of any method described herein, the mutation in a TSC gene results in the inactivation of the TSC gene, meaning that no transcription occurs from the gene and consequently no primary RNA transcript is produced from the gene.

In another embodiment of any method described herein, the mutation in a TSC gene results in an inactive protein, meaning that a transcribed and translated gene product from the mutated TSC gene is not functional as compared to the gene product from a non-mutated TSC gene. Analysis of a mutation in a TSC gene can be performed by clinical genetic testing for TSC, for example, by companies such as Athena Diagnostics.

Alternatively, for TSC loci gene analysis, the mutations in the TSC loci can be analyzed by any known genomic method in the art. For example, by single-strand conformation polymorphism analysis (SSCP) coupled with DNA sequencing as described by Galina D. et al., Am. J. Respir. Crit. Care Med.; 2001; 163:253-258; Hornigold N, et al., Oncogene; 1999; 18:2657-2661. Briefly, the coding exons of TSC1 or 2 are amplified by polymerase chain reaction (PCR) and the amplified PCR products are then analysed for variation on DNA gels without glycerol and with 5% glycerol. As a good number of TSC loci mutations result in chain-terminating, quantitative real-time (RT-PCR) assays can be used to analyze the amount of TSC1/2 mRNA as described in Kwiatkowska J. et al., Ann Hum Genet. 1998; 62:277-85. Alternatively, commercial kits are available, e.g., RT² qPCR Primer Assay for Human TSC1 and TSC2 respectively from SABIOSCIENCES™ catalog#PPH00244B-200 and PPH00245F. The PCR primers for the human TSC1 and TSC2 can be purchased from BIORAD. Alternatively, one skilled in the art can design PCR primers for the human TSC1 and TSC2 with the following information regarding the human TSC1 and TSC2 genes:

The gene symbol, TSC1 stands for the gene name tuberous sclerosis 1. Aliases for TSC1 include; KIAA0243, LAM, MGC86987, and TSC. The RefSeqs of TSC1 are NC_{—}000009.11; NG_{—}012386.1; NT_{—}035014.4. Ensembl: ENSG00000165699; Entrez: 7248; UniGene: Hs.370854.

The gene symbol, TSC2, stands for the gene name tuberous sclerosis 2. Aliases for TSC1 include F1143106, LAM, and TSC4. The RefSeqs of TSC2 are: NC_{—}000016.9; NG_{—}005895.1; NG_{—}008412.1; NG_{—}008617.1; and NT_{—}010393.16. Ensembl: ENSG00000103197; Entrez: 7249; UniGene: Hs.90303.

In one embodiment of any method described herein, the mutation in a TSC gene comprises a large gene deletion or insertion.

In one embodiment of any method described herein, the disease that is associated with a mutation in a TSC gene is LAM.

At least 50% of LAM is associated with a mutation in the TSC2 gene.

In one embodiment of any method described herein, the LAM is selected from the group consisting of tuberous sclerosis complex lymphangioleiomyomatosis (TSC-LAM) and non-heritable sporadic form lymphangioleiomyomatosis (S-LAM).

In one embodiment of any method described herein, the subject is mammal, e.g., human, cat, dog, rat, and mouse etc. In one embodiment of any method described herein, the subject is primate mammal. In one embodiment of any method described herein, the subject is non-primate mammal. In one embodiment of any method described herein, the subject is human.

In one embodiment of any method described herein, the subject is female.

In one embodiment of any method described herein, the female is of reproductive age. In one embodiment of any method described herein, the female subject of the reproductive age between 12-75 years old. In other embodiments of any method described herein, the female subject is between the ages of 12-70, 12-60, 12-65, 12-50, 12-55, 12-72, 15-20, 15-25, 15-30, 15-35, 15-40, 15-45, 20-25, 20-30, 20-35, 20-40, 20-45, 20-50, 25-30, 25-35, 25-40, 25-45, 25-50, 30-35, 30-40, 30-45, 30-50, 35-75, 35-40, 35-45, 35-50, 40-45, 40-50, 40-60, 40-65, 40-70, 40-75, 45-75, 45-65, 45-55, 45-60 and 45-50 years old, including any age ranges between 12-75.

In one embodiment of any method described herein, the female subject is in her 20s, in her 30s, in her 40s, in her 50s, in her 60s or in her 70s.

In one embodiment of any method described herein, the subject has LAM, TSC-LAM and S-LAM, or a disease associated with a mutation in a TSC gene.

In one embodiment of any method described herein, the subject is in remission from LAM, LAM, TSC-LAM and S-LAM, or a disease associated with a mutation in a TSC gene.

In one embodiment of any method described herein, the subject does not have any symptoms associated with LAM, LAM, TSC-LAM and S-LAM, or a disease associated with a mutation in a TSC gene.

The average age at diagnosis of LAM is about 35 years, after an average symptomatic period of 3-5 years. However, in general, the patients range from ages 12-75 years. Most women registered with LAM complain of dyspnea on exertion (51%). Symptoms of cough (6%), chest pain (5%), hemoptysis (5%), chyloptysis and wheezing were all considerably less common. Pneumothorax also occurs in 66% of LAM patients at some point in the course of their illness. First recurrences after an initial pneumothorax occurred in more than 70% of patients, and contralateral pneumothoraces were almost as common. Thus, once a LAM patient has had a pneumothorax, a second event is more likely than not. Chylothorax occurs in about 33% of LAM patients at some point in the illness. Angiomyolipomas are present in most patients with LAM, including 70-80% of patients with TSC-LAM and 40-50% of patients with LAM. Rarely, LAM presents as retroperitoneal masses or adenopathy which mimic lymphoma, ovarian or renal cancer, or other malignancy. Large lymph filled abdominal lymphangiomyomas have also been described, and may vary in size with gravitational influences in supine and erect patients.

Differential diagnosis of LAM, TSC-LAM and S-LAM, or a disease associated with a mutation in a TSC gene is known to one skilled in the art. For example, see Respiratory Medicine (2010) 104, S33-S41 by Simon R. Johnson. This reference is incorporated herein by reference in its entirety. In some embodiments, differential diagnosis of LAM, TSC-LAM and S-LAM, or a disease associated with a mutation in a TSC gene involves CT scan of chest, chest X-rays, genetic screening for mutation in at least one TSC gene, analysis of plasmas/sera VEGF-D levels, familial history, and a lung or other tissue biopsies.

As used herein, the term “differential diagnosis” with respect to LAM, TSC-LAM and S-LAM, or a disease associated with a mutation in a TSC gene refers to the final diagnosis of LAM, TSC-LAM and S-LAM, or a disease associated with a mutation in a TSC gen after all other respiratory ailments or ailments that can present similar symptoms have been systematically ruled out.

In one embodiment of any method described herein, the subject has at least one symptom known to be associated with LAM, TSC-LAM and S-LAM, or a disease associated with a mutation in a TSC gene. Non-limiting examples of symptoms associated with LAM, LAM, TSC-LAM and S-LAM, or a disease associated with a mutation in a TSC gene are dyspnea on exertion, cough, chest pain, hemoptysis, chyloptysis, shortness of breathe, wheezing, pneumothorax and chylothorax.

In one embodiment of any method described herein, the subject has been diagnosed with LAM, TSC-LAM and S-LAM, or a disease associated with a mutation in a TSC gene. A skilled physician will be able to diagnose LAM, LAM, TSC-LAM and S-LAM, or a disease associated with a mutation in a TSC gene based the known clinical symptoms, imaging techniques known in the art, lung biopsy, pulmonary function testing, and genetic analysis of the TSC loci in the subject.

In one embodiment of any method described herein, the subject has been diagnosed with an elevated level of LPC of at least 5% higher than a LPC level of a healthy subject or a predetermined reference LPC level derived from a population of healthy subjects.

In one embodiment of any method described herein, the subject has been diagnosed with an elevated level serum VEGF-D level of greater than 500 pg/mL. In other embodiments, the elevated level serum VEGF-D level is greater than 550 pg/mL, greater than 600 pg/mL, greater than 650 pg/mL, greater than 700 pg/mL, greater than 750 pg/mL, and greater than 800 pg/mL. Methods of diagnosing elevated level serum VEGF-D level are known in the art, e.g., in U.S. Pat. Nos. 7,811,776 and 8,058,018.

In one embodiment of any method described herein, the subject is asymptomatic for a common respiratory ailment.

In one embodiment of any method described herein, the subject has exhibited at least one common respiratory ailment that is known.

In another embodiment of any method described herein, the subject has not exhibited any common respiratory ailment that is known.

Non-limiting common respiratory ailments that are known include chronic obstructive pulmonary disease (COPD), asthma, bronchitis, common cold, sinusitis, congestion, lung cancer, pneumonia, emphysemapulmonary tuberculosis (TB), cystic fibrosis, diffuse interstitial diseases (alveolitis) and pneumothorax.

Current differential diagnosis of mesothelioma comprises elimination of common respiratory ailments, followed by confirmatory diagnosis by imaging techniques such as chest x-ray, CT scan or MRI, and by biopsy and cytological methods such as evidence of cancerous cells in pleural effusion or pleura.

In one embodiment of any method described herein, the subject does not have any symptoms selected from the group consisting of anemia, a blood clotting disorder, bowel obstruction, chest pain, persistent dry or raspy cough, coughing up blood (hemoptysis), shortness of breath (dyspnea), pain in the lower back or rib area, painful breathing, development of lumps under the skin on the chest, difficulty with swallowing (dysphagia), night sweats or fever, nausea, unexplained weight loss, fatigue, abdomen, pericardium, peritoneal and/or pleural effusion.

In one embodiment of any method described herein, the subject has at least one symptom selected from the group consisting of anemia, a blood clotting disorder, bowel obstruction, chest pain, persistent dry or raspy cough, coughing up blood (hemoptysis), shortness of breath (dyspnea), pain in the lower back or rib area, painful breathing, development of lumps under the skin on the chest, difficulty with swallowing (dysphagia), night sweats or fever, nausea, unexplained weight loss, fatigue, abdomen, pericardium, peritoneal and/or pleural effusion.

In one embodiment of any method described herein, the subject is being treated for LAM, TSC-LAM and S-LAM, or a disease associated with a mutation in a TSC gene. In another embodiment of any method described herein, the subject is receiving a treatment selected from the group consisting of hormone therapy, CK inhibition therapy, PLA2 inhibition therapy, mTORC1 inhibition therapy, PI3K inhibition therapy, oxygen therapy, pleurodesis, embolization, ablation or resection of angiomyolipomas, bronchodilator therapy, withdrawal from estrogen-containing medications, and thoracic duct ligation.

In one embodiment of any method described herein, the subject is being treated with an experimental drug in a clinical trial.

In one embodiment of any method described herein, the subject is being treated or administered the treatment before the first measurement LPC level.

In another embodiment of any method described herein, the subject is being treated or administered the treatment after the first measurement LPC level but before the second measurement LPC level.

In another embodiment of any method described herein, the subject is being treated or administered the treatment after the second measurement LPC level.

In one embodiment of any method described herein, the subject is being treated or administered the treatment before the imaging of carbon-11-labeled choline, fluorine 18-labeled choline or derivatives thereof in the subject at a first time point.

In another embodiment of any method described herein, the subject is being treated or administered the treatment after the first carbon-11-labeled choline, fluorine 18-labeled choline or derivatives but before the second carbon-11-labeled choline, fluorine 18-labeled choline or derivatives thereof in the subject.

In another embodiment of any method described herein, the subject is being treated or administered the treatment after the second carbon-11-labeled choline, fluorine 18-labeled choline or derivatives thereof in the subject at a first time point.

In one embodiment of any method described herein, the method further comprising selecting for a female subject. In another embodiment of any method described herein, the method further comprising selecting for a female subject of reproductive age.

In one embodiment of any method described herein, the method further comprising selecting for a female subject of reproductive age between 15-50 years old. In other embodiments of any method described herein, the method further comprising selecting for a female subject of reproductive age between the ages of 12-50, 12-55, 12-60, 15-20, 15-25, 15-30, 15-35, 15-40, 15-45, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, and 45-50 years old.

In another embodiment of any method described herein, the method further comprising selecting for a female subject of reproductive age in her 20s, in her 30s, in her 40s, in her 50s or in her 60s.

In one embodiment of any method described herein, the method further comprising selecting for a subject is in remission from LAM, LAM, TSC-LAM and S-LAM, or a disease associated with a mutation in a TSC gene.

In one embodiment of any method described herein, the method further comprising selecting a subject having LAM, TSC-LAM and S-LAM, or a disease associated with a mutation in a TSC gene. In another embodiment of any method described herein, the method further comprising selecting a subject who has been diagnosed with LAM, TSC-LAM and S-LAM, or a disease associated with a mutation in a TSC gene.

In one embodiment of any method described herein, the method further comprising selecting a subject who has been diagnosed with an elevated level of LPC of at least 5% higher than a LPC level of a healthy subject or a predetermined reference LPC level derived from a population of healthy subjects.

In one embodiment of any method described herein, the method further comprising selecting a subject who has been diagnosed with an elevated level serum VEGF-D level of greater than 500 pg/mL. In other embodiments of any method described herein, the method further comprising selecting a subject who has been diagnosed with an elevated level serum VEGF-D level greater than 550 pg/mL, greater than 600 pg/mL, greater than 650 pg/mL, greater than 700 pg/mL, greater than 750 pg/mL, and greater than 800 pg/mL.

In one embodiment of any method described herein, the method further comprising selecting a subject who is asymptomatic for a common respiratory ailment.

In one embodiment of any method described herein, the method further comprising selecting a subject who has exhibited at least one common respiratory ailment that is known.

In one embodiment of any method described herein, the method further comprising selecting a subject who has not exhibited any common respiratory ailment that is known.

In one embodiment of any method described herein, the method further comprising selecting a subject who does not have any symptom selected from the group consisting of anemia, a blood clotting disorder, bowel obstruction, chest pain, persistent dry or raspy cough, coughing up blood (hemoptysis), shortness of breath (dyspnea), pain in the lower back or rib area, painful breathing, development of lumps under the skin on the chest, difficulty with swallowing (dysphagia), night sweats or fever, nausea, unexplained weight loss, fatigue, abdomen, pericardium, peritoneal and/or pleural effusion.

In one embodiment of any method described herein, the method further comprising selecting a subject who has at least one symptom selected from the group consisting of anemia, a blood clotting disorder, bowel obstruction, chest pain, persistent dry or raspy cough, coughing up blood (hemoptysis), shortness of breath (dyspnea), pain in the lower back or rib area, painful breathing, development of lumps under the skin on the chest, difficulty with swallowing (dysphagia), night sweats or fever, nausea, unexplained weight loss, fatigue, abdomen, pericardium, peritoneal and/or pleural effusion.

In one embodiment of any method described herein, the method further comprising selecting a subject who is being treated for LAM, TSC-LAM and S-LAM, or a disease associated with a mutation in a TSC gene.

In another embodiment of any method described herein, the method further comprising selecting a subject who is receiving a treatment selected from the group consisting of hormone therapy, CK inhibition therapy, PLA2 inhibition therapy, mTORC1 inhibition therapy, PI3K inhibition therapy, oxygen therapy, pleurodesis, embolization, ablation or resection of angiomyolipomas, bronchodilator therapy, withdrawal from estrogen-containing medications, and thoracic duct ligation.

In one embodiment of any method described herein, the method further comprising selecting a subject who is being treated with an experimental drug in a clinical trial.

In one embodiment of any method described herein, the method further comprising determining that the subject having LAM has elevated LPC level in a biological sample obtained from the subject. In one embodiment of this method, the subject is a human.

In one embodiment of any method described herein, other additional treatments described herein are contemplated.

In one embodiment of any method described herein, non-limiting examples of treatments include hormone therapy, CK inhibition therapy, PLA2inhibitor therapy, mTORC1 inhibitor therapy, phosphatidylinositol 3-kinase (PI 3-kinases or PI3Ks) inhibitor therapy, oxygen therapy, pleurodesis, embolization, ablation or resection of angiomyolipomas; bronchodilator therapy, withdrawal from estrogen-containing medications, and thoracic duct ligation.

In one embodiment of any method described herein, the treatment is selected from the group consisting of hormone therapy, CK inhibition therapy, PLA2 inhibition therapy, mTORC1 inhibition therapy, and PI3K inhibition therapy.

Choline kinase (also known as CK,ChoK and Choline Phosphokinase) is an enzyme which catalyzes the first reaction in the choline pathway for phosphatidylcholine (PC) biosynthesis. This reaction involves the transfer of a phosphate group from ATP to choline in order to form phosphocholine. In mammalian cells, the enzyme exists as three isoforms, CKα-1, CKα-2 and CKβ. These isoforms are encoded by two separate genes, CHKA and CHKB and are only active in their homodimeric, heterodimeric and oligomeric forms.

Phospholipases A2 (PLA2s) EC 3.1.1.4 are enzymes that release fatty acids from the second carbon group of glycerol. This particular phospholipase specifically recognizes the sn-2 acyl bond of phospholipids and catalytically hydrolyzes the bond releasing arachidonic acid and lysophospholipids, of which lysophosphatidylcholines (LPC) is one. Upon downstream modification by cyclooxygenases, arachidonic acid is modified into active compounds called eicosanoids. Eicosanoids include prostaglandins and leukotrienes, which are categorized as inflammatory mediators.

Phosphatidylinositol 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). They are also known as phosphatidylinositol-3-kinases.

The mammalian target of rapamycin (mTOR) also known as mechanistic target of rapamycin or FK506 binding protein 12-rapamycin associated protein 1 (FRAP1) is a protein which in humans is encoded by the FRAP1 gene. mTOR is a serine/threonine protein kinase that regulates cell growth, cell proliferation, cell motility, cell survival, protein synthesis, and transcription. mTOR belongs to the phosphatidylinositol 3-kinase-related kinase protein family.

The PI3K/AKT/mTOR pathway is an intracellular signalling pathway important in apoptosis and hence cancer, e.g. breast cancer and non-small-cell lung cancer. mTOR signaling pathway.PI3K activation activates AKT which activates mTOR. (‘PDK1’ in the diagram is phosphoinositide-dependent kinase-1.) In many cancers this pathway is overactive reducing apoptosis and allowing proliferation. Thus some experimental cancer drugs aim to inhibit the signaling sequence at some point.

Lysophosphatidylcholines (LPC), also called lysolecithins, are a class of chemical compounds which are derived from phosphatidylcholines. They result from partial hydrolysis of phosphatidylcholines, which removes one of the fatty acid groups. The hydrolysis is generally the result of the enzymatic action of phospholipase A2. LPC is present as a minor phospholipid in the cell membrane (<3%) and in the blood plasma (8-12%). LPCs are quickly metabolized by lysophospholipase and LPC-acyltransferase, they last only shortly in vivo.

In one embodiment of any method described herein, the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, PI-103 and cyclophane.

In one embodiment of any method described herein, the PLA2 inhibitor is selected from the group consisting of darapladib, bromoenol lactone, varespladib (also known as A-001, previously LY315920 and S-5920) and palmitoyl trifluoromethyl ketone (PACOCF3).

In one embodiment of any method described herein, non-limiting examples of a PI3K inhibitor is selected from the group consisting of perifosine, wortmannin, demethoxyviridin, LY294002, CAL101, PX-866, BEZ235, SF1126, INK1117, IPI-145, GDC-0941, BKM120, XL147, XL765, Palomid 529, GSK1059615, ZSTK474, PWT33597, IC87114, TG100-115, CAL263, PI-103, GNE-477, CUDC-907, and AEZS-136.

In one embodiment of any method described herein, non-limiting examples of a mTOC1 inhibitor is selected from the group consisting of everolimus, temsirolimus and sirolimus.

In one embodiment of any method described herein, the biological samples obtained from the subjects are blood, serum, plasma or urine. In one embodiment of any method described herein, the biological samples obtained from the test subjects and the healthy subjects are the same. For example, both the biological samples obtained from the test subjects and the healthy subjects are plasmas. A skilled physician or technicians would able to collect these biological samples by known methods in the art.

Blood is collected into EDTA collection tubes and mixed by inversion. Samples are centrifuged (15 minutes at 1100 g) within four hours (or anywhere from 30 min −24 hrs) of blood collection to separate plasma. Plasma is collected using a transfer pipette and aliquots are transferred into cryovials for storage at −80° C. Analyses of lysophosphatidylcholines (LPC) are conducted using a liquid chromatography-mass spectrometry (LC-MS) system comprised of an 1100 Series pump (Agilent Technologies; Santa Clara, Calif.), an HTS PAL autosampler (Leap Technologies; Carrboro, N.C.), and a 4000 QTRAP mass spectrometer (AB SCIEX; Foster City, Calif.). Plasma samples (10 μL) are extracted for lipid analyses with 190 μL of isopropanol containing 1-dodecanoyl-2-tridecanoyl-sn-glycero-3-phosphocholine (Avanti Polar Lipids; Alabaster, Ala.). After centrifugation, supernatants are injected directly onto a 150×3.0 mm Prosphere HP C4 column (Grace, Columbia, Md.). The column is eluted isocratically with 80% mobile phase A (95:5:0.1 vol/vol/vol 10 mM ammonium acetate/methanol/acetic acid) for 2 minutes followed by a linear gradient to 80% mobile-phase B (99.9:0.1 vol/vol methanol/acetic acid) over 1 minute, a linear gradient to 100% mobile phase B over 12 minutes, then 10 minutes at 100% mobile-phase B. MS analyses are carried out using electrospray ionization and full scan MS over m/z 300-1100 in the positive ion mode. The ion spray voltage is 5.0 kV and the source temperature is 400° C. Data are processed using software from the instrument manufacturer (MultiQuant, AB SCIEX) to generate extracted ion chromatograms (XICs) of the [M+H]+ ion of each LPC and integrate the peak areas. The processed data are then exported to text file that may be opened using statistical analysis software.

In one embodiment, provided herein is a choline kinase (CK) inhibitor and/or a phospholipases A2 (PLA2) inhibitor for use in the treatment of LAM, TSC-LAM and S-LAM, or a disease associated with a mutation in a TSC gene in a subject.

In one embodiment, provided herein is a choline kinase (CK) inhibitor and/or a phospholipases A2 (PLA2) inhibitor for use in the manufacture of a medicament for the treatment of LAM, TSC-LAM and S-LAM, or a disease associated with a mutation in a TSC gene in a subject.

In one embodiment, provided herein is a PI3K inhibitor and/or an mTOR inhibitor for use in the treatment of LAM, TSC-LAM and S-LAM, or a disease associated with a mutation in a TSC gene in a subject.

In one embodiment, provided herein is a PI3K inhibitor and/or an mTORC1 inhibitor for use in the manufacture of a medicament for the treatment of LAM, TSC-LAM and S-LAM, or a disease associated with a mutation in a TSC gene in a subject.

In one embodiment of any treatment method described herein, the subject has an elevated level of LPC when compared with a predetermined reference LPC level.

In one embodiment of any treatment method described herein, the subject has increased uptake of isotope-label choline, increased isotope-label choline signal intensity and/or increased isotope-label choline location signals compared to the isotope-label choline imaging signals obtained from healthy subjects. In one embodiment, the isotope-label choline imaging is by PET or PET/CT scans.

In one embodiment of any treatment method described herein, the treatment of the subject with a CK, a PLA2, an mTOR and/or a PI3K inhibitor or combinations thereof results in a previously elevated LPC level in a biological sample from the subject being reduced by at least 5% compared to the a previously elevated LPC level or being reduced such that the elevated LPC level is at least 5% closer to a predetermined reference LPC level.

In practicing any of the methods described herein, it is contemplated that the skilled physician will use the temporal changes, if any, in the LPC levels or the isotope-labeled choline in the subject to monitor the progression, regression or recurrence of the LAM or disease in the subject, as well as to assess the efficacy of any treatment administered. In one embodiment of any treatment method described herein, the treatment comprises at least one routine screening of LPC levels and/or isotope-labeled choline in the subject while being treated, before treatment and/or after completion of a treatment regime.

Routine screening of LPC levels and/or isotope-labeled choline in the subject can be done, e.g., every month, every other month, every three months etc. when the subject is being treated and when no treatment is administered. A skilled physician would be able to determine the optimal frequency of screening required depending on whether the subject is being treated or not treated, and also depending on the stage of the LAM or disease in the subject among other factors considered. Accordingly, treatment may be administered between the routine screening of LPC levels and/or isotope-labeled choline in the subject such that at times, treatment may be administered before or after a measurement of LPC or imaging of isotope-labeled choline in the subject. The newest or latest results of the screening of LPC levels and/or isotope-labeled choline in the subject can be compared with the predetermined reference controls (i.e., those of healthy subjects), or compared with a previous screening results obtained for the same subject. If the newest or latest results of the screening are higher than the predetermined reference controls, this indicates continued presence of the LAM or the disease. If the newest or latest results of the screening are not any different from the predetermined reference controls, this indicates absence of the LAM or the disease. If the newest or latest results of the screening are lower than the predetermined reference controls, and the subject is still undergoing treatment, this indicates regression of the LAM or the disease. If the newest or latest results of the screening are lower than the predetermined reference controls, and the subject is still undergoing treatment, this indicates continued presence and progression of the LAM or the disease. If the newest or latest results of the screening are higher than the previous screening results of the same subject and the subject is still undergoing treatment, this indicates continued presence and progression of the LAM or the disease. If the newest or latest results of the screening are higher than the previous screening results of the same subject and the subject is not undergoing treatment, this indicates continued presence and progression of the LAM or the disease. If the newest or latest results of the screening are higher than the previous screening results of the same subject and the subject is in remission, this indicates re-appearance or recurrence of the LAM or the disease.

### Computer System and Computer Data Storage

In one embodiment, provided herein is a system comprising: a measuring module measuring a first measurement of LPC and a second measurement of LPC from biological samples obtained from a subject; a storage module configured to store data output from the measuring module; a comparison module adapted to compare the data stored on the storage module with a reference and/or control data, and to provide a retrieved content, and an output module for displaying the retrieved content for the user, wherein the retrieved content displays the comparison of the second LPC measurement with the first LPC measurement indicates that the subject has LAM or a disease associated with a TSC gene mutation, or indicates that the LAM or disease is on progressing, is regressing or there is no change in the status of the LAM or disease. In another embodiment, the measuring module measures subsequent measurement of LPCs. In another embodiment, the retrieved content displays the comparison of the first, second or any subsequent LPC measurement with the reference and/or control data, and indicates that the subject has LAM or a disease associated with a TSC gene mutation, or indicates that the LAM or disease is on progressing, is regressing or there is no change in the status of the LAM or disease.

In one embodiment, the measuring module is capable of measuring more than one measurement of LPC, e.g., two, three, four, five, six, seven, eight, nine, ten or even more.

In one embodiment, the reference and/or control data is the predetermined reference LPC level comprises the average LPC level measurements from a plurality of healthy human subjects without a disease associated with a mutation in a TSC gene as described herein.

In one embodiment, the reference and/or control data is the measured LPC levels of the same subject taken at an earlier time and were stored in the storage module for comparison purposes at a future time.

In one embodiment, provided herein is a system comprising: a measuring module measuring a first imaging signal of isotope-labeled choline and a second imaging signal of isotope-labeled choline in a subject; a storage module configured to store data output from the measuring module; a comparison module adapted to compare the data stored on the storage module with a reference and/or control data, and to provide a retrieved content, and an output module for displaying the retrieved content for the user, wherein the retrieved content displays the comparison of the second imaging signal with the first imaging indicates that the subject has LAM or a disease associated with a TSC gene mutation, or indicates that the LAM or disease is on progressing, is regressing or there is no change in the status of the LAM or disease.

In one embodiment, the measuring module is capable of measuring more than one imaging signal, e.g., two, three, four, five, six, seven, eight, nine, ten or even more.

In some embodiments, the imaging signals are signal intensity of the isotope-labeled choline in a subject and the location of the signals of isotope-labeled choline in a subject.

In one embodiment, the reference and/or control data is the imaging signals of the isotope-labeled choline in the same subject taken at an earlier time and were stored in the storage module for comparison purposes at a future time.

In one embodiment, the reference and/or control data is the imaging signals of the isotope-labeled choline in a healthy subject or a population of healthy subjects.

In one embodiment, provided herein is a system to facilitate the prognosis evaluation or diagnosis evaluation of LAM or a disease associated with a mutation in a TSC gene in a subject, the system comprising: a determination (measuring) module configured to receive and output the measured LPC levels from a biological sample obtained from a subject, or output imaging signals of the isotope-labeled choline; a storage module configured to store output information from the determination module; a comparison module adapted to compare the data stored on the storage module with reference and/or control data, and to provide a comparison content, and an output module for displaying the comparison content for the user. If the measured LPC levels are at least 5% higher than the reference which is the predetermined reference LPC level of healthy subjects, then the subject likely has LAM or the disease if the subject have not been previously diagnosed with LAM or the disease, or the subject has a relapse if the subject has been in remission and has minimally elevated or normal measured LPC levels at start of remission. Alternatively, if the measured test LPC levels are at least 5% higher than the measured LPC levels taken at earlier time points wherein the LPCs are from the same subject, and the earlier LPC levels are minimally elevated or normal measured LPC levels, then the comparison indicates that the subject likely has LAM or the disease if the subject have not been previously diagnosed with LAM or the disease, or the subject has a relapse if the subject has been in remission and has minimally elevated or normal measured LPC levels at start of remission. In addition, if the measured LPC levels taken at earlier time points were also elevated over the predetermined reference LPC level of healthy subjects, then if the measured test LPC levels are at least 5% higher than the measured LPC levels taken at earlier time points indicates the continued presence LAM or the disease, and that the LAM or disease is not regressing but rather progressing. Now if the subject is being treated, then the output data would indicate that the current treatment is ineffective.

In one embodiment, if the measured LPC levels are at least 5% lower than the reference which is the predetermined reference LPC level of healthy subjects, then the subject not likely to have LAM or the disease if the subject have not been previously diagnosed with LAM or the disease, or the subject does not have a relapse if the subject has been in remission and has minimally elevated or normal measured LPC levels at start of remission. Alternatively, if the measured test LPC levels are at least 5% lower than the measured LPC levels taken at earlier time points wherein the LPCs are from the same subject, and the earlier LPC levels are minimally elevated or normal measured LPC levels, then the comparison indicates that the subject does not likely have LAM or the disease if the subject have not been previously diagnosed with LAM or the disease, or the subject does not have a relapse if the subject has been in remission and has minimally elevated or normal measured LPC levels at start of remission. In addition, if the measured LPC levels taken at earlier time points were also elevated over the predetermined reference LPC level of healthy subjects, then if the measured test LPC levels are at least 5% lower than the measured LPC levels taken at earlier time points indicates that LAM LAM or the disease is regressing and is not progressing further. Now if the subject is being treated, then the output data would indicate that the current treatment is effective.

Accordingly, depending on the reference or control data to which the measured test LPC levels of the subject are compared with, an increase of at least 5% or a decrease of at least 5% can indicate likelihood of the presence, absence, regression or progression of LAM or the disease, whether as an initial diagnosis or as a relapse.

If the measured imaging signals of the isotope-labeled choline in a subject are at least 5% higher than the reference which is the imaging signals of the isotope-labeled choline in a subject taken at an earlier time point or the imaging signals of the isotope-labeled choline in a healthy subject or a population of healthy subjects, then the subject likely has LAM or the disease if the subject have not been previously diagnosed with LAM or the disease, or the subject has a relapse if the subject has been in remission and has minimally elevated or normal measured LPC levels at start of remission.

In one embodiment, provided herein is a computer readable storage medium comprising: a storing data module containing data from a biological sample obtained from a subject comprising a detectable signal from an assay indicating the measured level of at least one LPC or imaging signals of isotope-labeled choline described herein; a comparison module that compares the data stored on the storing data module with a reference data and/or control data, and to provide a comparison content, and an output module displaying the comparison content for the user.

Embodiments of disclosed herein can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media #30 can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present technology. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments disclosed herein include at minimum a measuring module #40, a storage module #30, a comparison module #80, and an output module #110. The functional modules can be executed on one, or multiple, computers, or by using one, or multiple, computer networks. The measuring module has computer executable instructions to provide for handling the signal from the measured LPC levels or the signals from imaging of isotope-labeled choline.

In one embodiment, the measuring module #40 can comprise any system for detecting a signal representing the presence of LPC levels in the biological samples obtained from test subjects or healthy subjects, or imaging signals of the isotope-labeled choline in test subjects or healthy subjects. In one embodiment, the measuring module #40 is a LC-MS. In another embodiment, the measuring module #40 is a GC-CIMS. In other embodiments, the measuring module #40 is a PET scanner or a PET/CT scanner.

In one embodiment, the measuring module #40 can comprise any system for detecting a signal representing the signal intensity and location signals of isotope-labeled choline in a subject.

The information determined in the determination system can be read by the storage module #30. As used herein the “storage module” is intended to include any suitable computing or processing apparatus or other device configured or adapted for storing data or information. Examples of electronic apparatus suitable for use with the present technology include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon measured LPC level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment the reference data stored in the storage module to be read by the comparison module is e.g., the predetermined reference LPC level comprising an average LPC level measurement from a plurality of healthy subjects without LAM or any or disease associated with a mutation in a TSC gene or a measured LPC level obtained from the same subject at a prior earlier time point using the measuring module #40. Alternatively, the reference data stored in the storage module is the signal intensity and location signals of isotope-labeled choline in a test subject (ie., a subject suspected of having LAM or the disease described herein) at a prior earlier time point or a healthy subject or a plurality of healthy subjects without LAM or any or disease associated with a mutation in a TSC gene.

The “comparison module” #80 can use a variety of available software programs and formats for the comparison operative to compare the measured LPC level to reference measured LPC levels of an earlier time point and/or stored reference data, or the imaging signals of isotope-labeled choline obtained at an earlier time point from the same subject. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information the presence/absence of LAM or a disease associated with a mutation in at least a TSC gene in a subject, efficacy of drug or treatment in a subject, and/or method for treating of a subject.

The comparison module, or any other module of systems described, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank™ or Swiss Pro World Wide Web site). Thus, in a particular preferred embodiment of the present technology, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using an output module #110.

The content based on the comparison result may be a measured test LPC level compared to the predetermined reference LPC level or compared to a measured LPC level obtained from the same subject at a prior earlier time point.

The content based on the comparison result may be an imaging signal compared to the predetermined reference imaging signal or compared to an imaging signal obtained from the same subject at a prior earlier time point.

In one embodiment of the system, the content based on the comparison result is displayed on a computer monitor #120. In one embodiment of the system, the content based on the comparison result is displayed through printable media #130, #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the technology can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

Therefore, provided herein are systems (and computer readable media for causing computer systems) to perform the methods described herein (by detecting the presence of elevated LPC levels or imaging signals described herein); for diagnosing LAM or a disease associated with a mutation in at least one TSC gene; for assessing drug or treatment efficacy of LAM a disease associated with a mutation in at least one TSC gene; or for assessing treatment prognosis of LAM or a disease associated with a mutation in at least one TSC gene in a subject.

Systems and computer readable media described herein are merely illustrative embodiments described herein for assessing disease activity, diagnosing, prognosing and assessing drug or treatment efficacy in a subject, and are not intended to limit the scope of the technology. Variations of the systems and computer readable media described herein are possible and are intended to fall within the scope of the technology.

The modules of the machine, or those used in the computer readable medium, may assume numerous configurations. For example, function may be provided on a single machine or distributed over multiple machines.

### Formulation and Application

In one embodiment, the CK, PLA2, mTOR and/or PI3K inhibitor or combinations thereof are delivered in a pharmaceutically acceptable carrier.

In some embodiments, provided herein are therapeutic compositions comprising a CK, a PLA2, an mTOR and/or a PI3K inhibitor or combinations thereof for the treatment of LAM, TSC-LAM and S-LAM, or a disease associated with a mutation in a TSC gene in a subject.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration. Additional carrier agents, such as liposomes, can be added to the pharmaceutically acceptable carrier.

Therapeutic compositions contain a physiologically tolerable carrier together with at least a CK, a PLA2, an mTOR and/or a PI3K inhibitor or combinations thereof as described herein, dissolved or dispersed therein as an active ingredient. In one embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Compositions can be prepared as injectable either as liquid solutions or suspensions, however, solid forms suitable for solution, or suspensions; in liquid prior to use can also be prepared. The preparation can also be emulsified or presented as a liposome composition. The CK, PLA2, mTOR and/or PI3K inhibitor or combinations thereof can also be conjugated with lipids, e.g., amphipathic lipids, for stability and delivery purposes. The conjugation bonds are reversible and are broken or dissolved when the CK, PLA2, mTOR and/or PI3K inhibitor or combinations thereof are delivered to target destination. Alternatively, the CK, PLA2, mTOR and/or PI3K inhibitor or combinations thereof described herein can be prepared as a solid or semi-solid or emulsion in suppository, e.g., as microspheres. The microspheres can be inserted as a solid into or targeted to a solid tumor. The CK, PLA2, mTOR and/or PI3K inhibitor or combinations thereof described herein can be mixed with excipients which are pharmaceutically acceptable and compatible with the active ingredient and in amounts suitable for use in the therapeutic methods described herein. Specifically contemplated pharmaceutical compositions are CK, PLA2, mTOR and/or PI3K inhibitor or combinations thereof in a preparation for delivery as described herein above, or in references cited and incorporated herein in that section. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising the CK, PLA2, mTOR and/or PI3K inhibitor or combinations thereof described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of CK, PLA2, mTOR and/or PI3K inhibitor or combinations thereof used in the methods described herein that will be effective in the treatment of a particular disorder or condition will depend on the nature of the disorder or condition, and can be determined by standard clinical techniques.

Routes of administration include, but are not limited to, direct injection, intradermal, intramuscular, intraperitoneal, intravenous, subcutaneous, intrauterine and oral routes. The CK, PLA2, mTOR and/or PI3K inhibitor or combinations thereof described herein can be administered by any convenient route, for example by infusion, intravenous injection, suppository or bolus injection, by absorption through epithelial or mucocutaneous linings (e.g., oral mucosa, rectal and intestinal mucosa, etc.) and may be administered together with other biologically active agents. Administration can be systemic or local.

The precise dose and formulation to be employed depends upon the potency of the CK, PLA2, mTOR and/or PI3K inhibitor or combination thereof described herein, and depends on the amounts large enough to produce the desired effect, e.g., a reduction in size and/or growth of the tumors in the subject. The dosage should not be so large as to cause unacceptable adverse side effects. Generally, the dosage will vary with the type CK, PLA2, mTOR and/or PI3K inhibitor, and with the age, condition, and size of the tumors in the subject are also considered. Dosage and formulation of the CK, PLA2, mTOR and/or PI3K inhibitor will also depend on the route of administration, and the mass and number of tumors in the subject, and should be decided according to the judgment of the practitioner and each subject's circumstances. Effective doses can be extrapolated from dose-response curves derived from in vitro or animal model test systems.

The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

Administration of the doses recited above can be repeated for a limited period of time. In some embodiments, the doses are given once a day, or multiple times a day, for example but not limited to three times a day. In one embodiment, the doses recited above are administered daily for several weeks or months. The duration of treatment depends upon the subject's clinical progress and responsiveness to therapy, e.g., shrinkage of tumor sizes. Continuous, relatively low maintenance doses are contemplated after an initial higher therapeutic dose. As exemplary, the CK, PLA2, mTOR and/or PI3K inhibitor or combinations thereof and a pharmaceutically acceptable carrier can be formulated for intravenous application by injection in the subject.

Efficacy testing can be performed during the course of treatment using the methods described herein, e.g., ultrasound, MRI and CT to monitor the shrinkage in size of the tumors in the treated subject, assessing pulmonary function testing, exercise function testing, or any of the assessment or monitoring methods described herein. Such function testing are well known in the art. Pulmonary Function Testing (PFT) is a complete evaluation of the respiratory system including patient history, physical examinations, chest x-ray examinations, arterial blood gas analysis, and tests of pulmonary function. The primary purpose of pulmonary function testing is to identify the severity of pulmonary impairment. Pulmonary function testing has diagnostic and therapeutic roles and helps clinicians answer some general questions about patients with lung disease. PFT's are normally performed by a specialist technician. The two most common exercise function tests are the 6-min walk test and full cardiopulmonary exercise testing.

A decrease in size of the tumors during and after treatment indicates that the treatment is effective in reducing tumor size. Measurements of the degree of severity of a number of symptoms associated with proliferation of the LAM cells or cells having a mutation in a TSC gene are also noted prior to the start of a treatment and then at later specific time period after the start of the treatment. A skilled physician will be able to ascertain the tumor sizes, cysts lesions and related symptoms by known methods in the art and those described herein.

The present invention can be defined in any of the following numbered paragraphs:
[1] A choline kinase (CK) inhibitor and/or a phospholipases A2 (PLA2) inhibitor for use in the treatment of lymphangioleiomyomatosis (LAM) or a disease associated with a mutation in a TSC gene in a subject, wherein an elevated level of lysophosphatidylcholine (LPC) in a biological sample from the subject is reduced.
[2] A choline kinase (CK) inhibitor and/or a phospholipases A2 (PLA2) inhibitor for use in the manufacture of a medicament for the treatment of lymphangioleiomyomatosis (LAM), or a disease associated with a mutation in a TSC gene in a subject, wherein an elevated level of lysophosphatidylcholine (LPC) in a biological sample from the subject is reduced.
[3] The use of any one of paragraphs 1-2, wherein the subject has been previously diagnosed as having elevated LPC levels over a predetermined reference LPC level.
[4] The use of any one of paragraphs 1-3, wherein the reduction is at least 5% compared to the previously diagnosed as having elevated LPC level.
[5] The use of any one of paragraphs 1-4, wherein the reduction is at least 5% compared to the predetermined reference LPC level.
[6] The use of any one of paragraphs 3-5, wherein the predetermined reference LPC level comprises the average LPC level measurements from a plurality of healthy human subjects.
[7] The use of any one of paragraphs 1-6, wherein the subject is female.
[8] The use of paragraph 7, wherein the female is of reproductive age.
[9] The use of any one of paragraphs 1-8, wherein the mutation is in the TSC1 gene and/or TSC 2 gene.
[10] The use of any one of paragraphs 1-9, wherein the mutation results in no protein production or no functional protein.
[11] The use of any one of paragraphs 1-10, wherein the disease is LAM.
[12] The use of paragraph 11, wherein LAM is selected from the group consisting of tuberous sclerosis complex lymphangioleiomyomatosis (TSC-LAM) and non-heritable sporadic form lymphangioleiomyomatosis (S-LAM).
[13] The use of any one of paragraphs 1-12, wherein the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, P1-103 and cyclophane.
[14] The use of any one of paragraphs 1-13, wherein the PLA2 inhibitor is selected from the group consisting of darapladib, bromoenol lactone, varespladib and palmitoyl trifluoromethyl ketone.
[15] A method of treating lymphangioleiomyomatosis (LAM) or a disease associated with a mutation in a tuberous sclerosis complex (TSC) gene in a subject comprising administering a therapeutically effective amount of a choline kinase (CK) inhibitor and/or a therapeutically effective amount of a phospholipases A2 (PLA2) inhibitor to the subject in need thereof, wherein an elevated level of lysophosphatidylcholine (LPC) in a biological sample from the subject is reduced or is at least 5% closer to a predetermined reference LPC level.
[16] A method of treating lymphangioleiomyomatosis (LAM) or a disease associated with a mutation in a tuberous sclerosis complex (TSC) gene in a subject comprising
   a. diagnosing a subject as having LAM by a method comprising measuring the level of lysophosphatidylcholine (LPC) in a biological sample obtained from the subject, wherein an elevated LPC level in the sample compared to a predetermined reference LPC level is indicative of LAM in the subject; and
   b. administering a therapeutically effective amount of a choline kinase (CK) inhibitor and/or a therapeutically effective amount of a phospholipases A2 (PLA2) inhibitor to the subject in need thereof, wherein an elevated level of LPC in a biological sample from the subject is reduced, or is at least 5% closer to a predetermined reference LPC level.
[17] The method of paragraph 16 further comprising selecting a subject having LAM or a disease associated with a mutation in a TSC gene.
[18] The method of paragraph 16 or 17 further comprising determining that the subject having LAM has elevated LPC level in a biological sample obtained from the subject.
[19] The method of any one of paragraphs 16-18, wherein the mutation is in the TSC1 gene and/or TSC 2 gene.
[20] The method of any one of paragraphs 16-19, wherein the mutation results in no protein production or no functional protein.
[21] The method of any one of paragraphs 16-20, wherein the disease is LAM.
[22] The method of paragraph 21, wherein the LAM is selected from the group consisting of tuberous sclerosis complex lymphangioleiomyomatosis (TSC-LAM) and non-heritable sporadic form lymphangioleiomyomatosis (S-LAM).
[23] The method of any one of paragraphs 16-22, wherein the predetermined reference LPC level comprises an average LPC level measurement from a plurality of healthy subjects without LAM or any or disease associated with a mutation in a TSC gene, wherein an elevated level of LPC in the sample compared to the predetermined reference LPC level is diagnostic of the presence of LAM or the disease associated with a mutation in a TSC gene respectively.
[24] The method of any one of paragraphs 16-23, wherein the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, PI-103 and cyclophane.
[25] The method of any one of paragraphs 16-24, wherein the PLA2 inhibitor is selected from the group consisting of darapladib, bromoenol lactone, varespladib and palmitoyl trifluoromethyl ketone.
[26] A method of treating lymphangioleiomyomatosis (LAM) or a disease associated with a mutation in a tuberous sclerosis complex (TSC) gene in a subject, the method comprising:
   a. measuring the level of lysophosphatidylcholine (LPC) in a first biological sample obtained from the subject at a first time point;
   b. measuring the level of LPC in a second biological sample obtained from the subject at a second time point, wherein the first and second biological samples are of the same type, and wherein the first and second time are in chronological order; and
   c. comparing the first measurement and the second measurement of LPC wherein the results of the comparison determines the course of LAM or the disease in the subject.
[27] The method of claim 26 further comprising (d) deciding a course of action for the subject based upon the results of the comparison in step (c) wherein the course of action is selected from the group consisting of maintained no treatment, terminating current treatment, modify current treatment, and maintained current treatment; and (e) executing the course of action decided.
[28] A method of treating lymphangioleiomyomatosis (LAM) or a disease associated with a mutation in a tuberous sclerosis complex (TSC) gene in a subject, the method comprising:
   a. administering to the subject a treatment for treating LAM or the disease;
   b. measuring the level of lysophosphatidylcholine (LPC) in a first biological sample obtained from the subject at a first time point after start of the treatment;
   c. measuring the level of LPC in a second biological sample obtained from the subject at a second time point after start of treatment, wherein the first and second biological samples are of the same type, and wherein the first and second time are in chronological order; and
   d. comparing the first measurement and the second measurement of LPC wherein the results of the comparison determines the course of LAM or disease in the subject.
[29] A method of treating lymphangioleiomyomatosis (LAM) or a disease associated with a mutation in a tuberous sclerosis complex (TSC) gene in a subject, the method comprising:
   a. measuring the level of lysophosphatidylcholine (LPC) in a first biological sample obtained from the subject at a first time point prior to start of a treatment;
   b. administering to the subject a treatment for treating LAM or the disease;
   c. measuring the level of LPC in a second biological sample obtained from the subject at a second time point after start of the treatment, wherein the first and second biological samples are of the same type, and wherein the first and second time are in chronological order; and
   d. comparing the first measurement and the second measurement of LPC wherein the results of the comparison determines the course of LAM or disease in the subject.
[30] The method of claim 28 or 29 further comprising (e) deciding a course of action for the subject based upon the results of the comparison in step (d) wherein the course of action is selected from the group consisting of terminating current treatment, modify current treatment, and maintained current treatment; and (f) executing the course of action decided.
[31] A method of assessing the efficacy of a drug treatment in a subject, the method comprising:
   a. administering to the subject a treatment, wherein the subject has lymphangioleiomyomatosis (LAM) or a disease associated with a mutation in a tuberous sclerosis complex (TSC) gene;
   b. measuring the level of lysophosphatidylcholine (LPC) in a first biological sample obtained from the subject at a first time point;
   c. measuring the level of LPC in a second biological sample obtained from the subject at a second time point, wherein the first and second biological samples are of the same type, and wherein the first and second time are in chronological order; and
   d. comparing the first measurement and the second measurement of LPC wherein the results of the comparison determines whether the drug treatment is effective in the subject.
[32] A method of assessing the efficacy of a drug treatment in a subject, the method comprising:
   a. measuring the level of lysophosphatidylcholine (LPC) in a first biological sample obtained from the subject at a first time point, wherein the subject has lymphangioleiomyomatosis (LAM) or a disease associated with a mutation in a tuberous sclerosis complex (TSC) gene;
   b. administering to the subject a treatment;
   c. measuring the level of LPC in a second biological sample obtained from the subject at a second time point, wherein the first and second biological samples are of the same type, and wherein the first and second time are in chronological order;
   d. comparing the first measurement and the second measurement of LPC wherein the results of the comparison determines whether the drug treatment is effective in the subject.
[33] The method of paragraph 31 or 32 further comprising deciding a course of action for the subject based upon the results of the comparison in step (d) wherein the course of action is selected from the group consisting of maintained current drug application, terminating current drug application, and modify current drug application.
[34] The method of any one of paragraphs 26-33 further comprising selecting a subject having LAM or a disease associated with a mutation in a TSC gene.
[35] The method of any one of paragraphs 26-34, wherein the mutation is in the TSC1 gene and/or TSC 2 gene.
[36] The method of any one of paragraphs 26-35, wherein the mutation results in no protein production or no functional protein.
[37] The method of any one of paragraphs 26-36, wherein the disease is lymphangioleiomyomatosis (LAM).
[38] The method of any one of paragraphs 26-37, wherein LAM is selected from the group consisting of tuberous sclerosis complex lymphangioleiomyomatosis (TSC-LAM) and non-heritable sporadic form lymphangioleiomyomatosis (S-LAM).
[39] The method of any one of paragraphs 26-38, wherein the subject is female.
[40] The method of paragraph 39, wherein the female is of reproductive age.
[41] The method of any one of paragraphs 26-40, wherein the treatment is selected from the group consisting of hormone therapy, choline kinase (CK) inhibition therapy, phospholipase A2 (PLA2) inhibition therapy, mTORC1 inhibition therapy, phosphatidylinositol 3-kinase (PI 3-kinase or PI3K) inhibition therapy, oxygen therapy, pleurodesis, embolization, ablation or resection of angiomyolipomas; bronchodilator therapy, withdrawal from estrogen-containing medications, and thoracic duct ligation.
[42] The method of any one of paragraphs 26-41, wherein the treatment is selected from the group consisting of hormone therapy, CK inhibition therapy, PLA2 inhibition therapy, mTORC1 inhibition therapy, and PI3K inhibition therapy.
[43] The method of paragraphs 41 or 42, wherein the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, PI-103 and cyclophane.
[44] The method of paragraphs 41 or 42, wherein the PLA2 inhibitor is selected from the group consisting of darapladib, bromoenol lactone, varespladib and palmitoyl trifluoromethyl ketone.
[45] The method of paragraphs 41 or 42, wherein the PI3K inhibitor is selected from the group consisting of perifosine, wortmannin, demethoxyviridin, LY294002, CAL101, PX-866, BEZ235, SF1126, INK1117, IPI-145, GDC-0941, BKM120, XL147, XL765, Palomid 529, GSK1059615, ZSTK474, PWT33597, IC87114, TG100-115, CAL263, PI-103, GNE-477, CUDC-907, and AEZS-136.
[46] The method of paragraphs 41 or 42, wherein the mTORC1 inhibitor is selected from the group consisting of everolimus, temsirolimus and sirolimus.
[47] The method of any one of paragraphs 16-46, wherein the LPC is measured by liquid chromatography coupled to mass spectrometry (LC-MS), enzymatic measurements, or gas chromatography coupled to chemical ionization mass spectrometry (GC-CIMS).
[48] The method of any one of paragraphs 16-47, wherein the LPC measured is selected from the selected from the group consisting of: C14:0 LPC; C16:0 LPC; C16:1 LPC; C18:0 LPC; C18.1 LPC; C18:3 LPC; C18:2 LPC; C20:3 LPC; C20:4 LPC; and C22:6 LPC.
[49] The method of any one of paragraphs 16-48, wherein the biological samples are serum, plasma or urine.
[50] The method of any one of paragraphs 16-49, wherein the predetermined reference LPC level comprises an average LPC level measurement from a plurality of healthy subjects.
[51] The method of any one of paragraphs 26-50, wherein the first time point is selected for the group consisting of prior to an application of a treatment, during the course of a first treatment, the course of a second treatment, and the course of a subsequent treatment.
[52] The method of any one of paragraphs 26-51, wherein the second time point is selected for the group consisting of prior to an application of a treatment, during the course of a first treatment, the course of a second treatment, and the course of a subsequent treatment.
[53] The method of any one of paragraphs 26-52, wherein course of LAM or the disease in the subject in selected from the group consisting of no further progression, continued progression and regression.
[54] The method of paragraph 53, wherein both the first and second measurements of LPC are prior to an application of a treatment, and wherein the second measurement of LPC is the same or is no more than 5% over the first measurement of LPC indicates that there is no further progression of LAM in the subject, and the course of action is to maintain no treatment in the subject.
[55] The method of paragraph 54, wherein both the first and second measurements of LPC are prior to an application of a treatment, and wherein the second measurement of LPC is at least 5% over or higher the first measurement of LPC indicates that there is progression of LAM in the subject, and the course of action is to initiate a treatment in the subject.
[56] The method of paragraph 55, wherein both the first and second measurements of LPC are during the course of a first or second or subsequent treatment, wherein the second measurement of LPC is during the course of the treatment is at least lower by 5% compared to the first measurement of LPC indicates regression of LAM in the subject, and that the current treatment is effective and is to be maintained.
[57] The method of paragraph 55, wherein both the first and second measurements of LPC are during the course of a first or second or subsequent treatment, wherein the second measurement of LPC is during the course of the treatment remains the same as or is higher by at least 5% over the first measurement of LPC indicates continued progression of LAM in the subject, and that the current treatment is ineffective and is to be terminated or modified.
[58] The method of paragraph 55, wherein the first measurement of LPC is prior to an application of a treatment and the second measurement of LPC is during the course of a first or second or subsequent treatment, wherein the second measurement of LPC during the course of the treatment is the same as or elevated at least 5% over or higher the first measurement of LPC prior to start of the treatment indicates continued progression of LAM in the subject, and that the current treatment is ineffective and is to be terminated or modified.
[59] The method of paragraph 55, wherein the first measurement of LPC is prior to an application of a treatment and the second measurement of LPC is during the course of the first or second or subsequent treatment, wherein the second measurement of LPC is at least lower by 5% compared to the first measurement of LPC indicates regression of LAM in the subject, and that the current treatment is effective and is to be maintained.
[60] The method of paragraph 55, wherein the first measurement of LPC is a first treatment and the second measurement of LPC is during the course of the second or subsequent treatment, wherein the second measurement of LPC is the same as or elevated at least 5% over the first measurement of LPC indicates continued progression of LAM in the subject, and that the second or subsequent treatment is ineffective and is to be terminated or modified.
[61] The method of paragraph 55, wherein the first measurement of LPC is a first treatment and the second measurement of LPC is during the course of the second or subsequent treatment, wherein the second measurement of LPC is at least lower by 5% compared to the first measurement of LPC indicates regression of LAM in the subject, and that the second or subsequent treatment is effective and is to be maintained.
[62] A method of treating lymphangioleiomyomatosis (LAM) or a disease associated with a mutation in a tuberous sclerosis complex (TSC) gene in a subject, the method comprising:
   a. imaging the density and location of carbon-11-labeled choline, fluorine 18-labeled choline or derivatives thereof in the subject at a first time;
   b. imaging the density and location of carbon-11-labeled choline, fluorine 18-labeled choline or derivatives thereof in the subject at a second time, wherein the first and second time are in chronological order; and
   c. comparing the first imaging and the second imaging, wherein the results of the comparison determines the course of LAM or disease in the subject.
[63] The method of paragraph 62 further comprising (d) deciding a course of action for the subject based upon the results of the comparison in step (c) wherein the course of action is selected from the group consisting of maintained no treatment, terminating current treatment, modify current treatment, and maintained current treatment; and (e) executing the course of action decided.
[64] A method of treating lymphangioleiomyomatosis (LAM) or a disease associated with a mutation in a tuberous sclerosis complex (TSC) gene in a subject, the method comprising:
   a. administering to the subject a treatment;
   b. imaging the density and location of carbon-11-labeled choline, fluorine 18-labeled choline or derivatives thereof in the subject at a first time;
   c. imaging the density and location of carbon-11-labeled choline, fluorine 18-labeled choline or derivatives thereof in the subject at a second time, wherein the first and second time are in chronological order; and
   d. comparing the first imaging and the second imaging, wherein the results of the comparison determines the course of LAM or disease in the subject.
[65] A method of treating lymphangioleiomyomatosis (LAM) or a disease associated with a mutation in a tuberous sclerosis complex (TSC) gene in a subject, the method comprising:
   a. imaging the density and location of carbon-11-labeled choline, fluorine 18-labeled choline or derivatives thereof in the subject at a first time;
   b. administering to the subject a treatment;
   c. imaging the density and location of carbon-11-labeled choline, fluorine 18-labeled choline or derivatives thereof in the subject at a second time, wherein the first and second time are in chronological order; and
   d. comparing the first imaging and the second imaging, wherein the results of the comparison determines the course of LAM or disease in the subject.
[66] The method of paragraph 64 or 65 further comprising (e) deciding a course of action for the subject based upon the results of the comparison in step (d) wherein the course of action is selected from the group consisting of terminating current treatment, modify current treatment, and maintained current treatment; and (f) executing the course of action decided.
[67] The method of any one of paragraphs 62-66 further comprising selecting a subject having LAM or a disease associated with a mutation in a TSC gene.
[68] The method of any one of paragraphs 62-67, wherein the mutation is in the TSC1 gene and/or TSC 2 gene.
[69] The method of any one of paragraphs 62-68, wherein the mutation results in no protein production or no functional protein.
[70] The method of any one of paragraphs 62-69, wherein the disease is lymphangioleiomyomatosis (LAM).
[71] The method of any one of paragraphs 62-70, wherein LAM is selected from the group consisting of tuberous sclerosis complex lymphangioleiomyomatosis (TSC-LAM) and non-heritable sporadic form lymphangioleiomyomatosis (S-LAM).
[72] The method of any one of paragraphs 62-71, wherein the subject is female.
[73] The method of paragraph 72, wherein the female is of reproductive age.
[74] The method of any one of paragraphs 62-73, wherein the treatment is selected from the group consisting of hormone therapy, choline kinase (CK) inhibition therapy, phospholipase A2 (PLA2) inhibition therapy, mTORC1 inhibition therapy, phosphatidylinositol 3-kinase (PI 3-kinase or PI3K) inhibition therapy, oxygen therapy, pleurodesis, embolization, ablation or resection of angiomyolipomas; bronchodilator therapy, withdrawal from estrogen-containing medications, and thoracic duct ligation.
[75] The method of any one of paragraphs 62-74, wherein the treatment is selected from the group consisting of hormone therapy, CK inhibition therapy, PLA2 inhibition therapy, mTORC1 inhibition therapy, and PI3K inhibition therapy.
[76] The method of paragraphs 74 or 75, wherein the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, PI-103 and cyclophane.
[77] The method of paragraphs 74 or 75, wherein the PLA2 inhibitor is selected from the group consisting of darapladib, bromoenol lactone, varespladib and palmitoyl trifluoromethyl ketone.
[78] The method of paragraphs 74 or 75, wherein the PI3K inhibitor is selected from the group consisting of perifosine, wortmannin, demethoxyviridin, LY294002, CAL101, PX-866, BEZ235, SF1126, INK1117, IPI-145, GDC-0941, BKM120, XL147, XL765, Palomid 529, GSK1059615, ZSTK474, PWT33597, IC87114, TG100-115, CAL263, PI-103, GNE-477, CUDC-907, and AEZS-136.
[79] The method of paragraphs 74 or 75, wherein the mTORC1 inhibitor is selected from the group consisting of everolimus, temsirolimus and sirolimus.
[80] The method of any one of paragraphs 62-79, wherein the first time point is selected for the group consisting of prior to an application of a treatment, during the course of a first treatment, the course of a second treatment, and the course of a subsequent treatment.
[81] The method of any one of paragraphs 62-80, wherein the second time point is selected for the group consisting of prior to an application of a treatment, during the course of a first treatment, the course of a second treatment, and the course of a subsequent treatment.
[82] The method of any one of paragraphs 62-81, wherein course of LAM or the disease in the subject in selected from the group consisting of no further progression, continued progression and regression.
[83] The method of any one of paragraphs 62-82, wherein the second imaging has lower density and location signal by at least 5% compared to the first imaging indicates regression of LAM in the subject, and that the course of action is to be maintained.
[84] The method of any one of paragraphs 62-82, wherein the second imaging density and location signals remain the same as or are higher by at least 5% over the first imaging indicates progression of LAM in the subject, and that the course of action is to be terminated or modified.

This invention is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the invention described herein. Such equivalents are intended to be encompassed by the following claims.

### Example

### Materials and Methods

Liquid Chromatography-Mass Spectrometry (LC-MS) was used to perform metabolomic profiling on the plasma of 14 Lymphangioleiomyomatosis (“LAM”) patients, compared with 9 healthy female controls. All of these samples were collected at the Brigham and Women's Hospital.

Data were acquired using an LC-MS system comprised of an 1100 Series pump (Agilent Technologies; Santa Clara, Calif.), an HTS PAL autosampler (Leap Technologies; Carrboro, N.C.), and a 4000 QTRAP mass spectrometer (AB SCIEX; Foster City, Calif.). Plasma samples (10 μL) were extracted using 190 μL of isopropanol containing 1-dodecanoyl-2-tridecanoyl-sn-glycero-3-phosphocholine (Avanti Polar Lipids; Alabaster, Ala.). After centrifugation, supernatants were injected directly onto a 150×3.0 mm Prosphere HP C4 column (Grace, Columbia, Md.). The column was eluted isocratically with 80% mobile phase A (95:5:0.1 vol/vol/vol 10 mM ammonium acetate/methanol/acetic acid) for 2 minutes followed by a linear gradient to 80% mobile-phase B (99.9:0.1 vol/vol methanol/acetic acid) over 1 minute, a linear gradient to 100% mobile phase B over 12 minutes, then 10 minutes at 100% mobile-phase B. MS analyses were carried out using electrospray ionization and Q1 scans in the positive ion mode over m/z 300-1100. The ion spray voltage is 5.0 kV and the source temperature is 400° C. Data were processed using MultiQuant 1.2 software (AB SCIEX) and integrated lipid peak areas were exported to a text file and statistics were performed using Excel (Microsoft). To normalize the data relative to control, the peak area of each LPC in each sample was divided by the mean peak area of the control group. A two-tailed Student's t test was used to compare means between cases and controls.

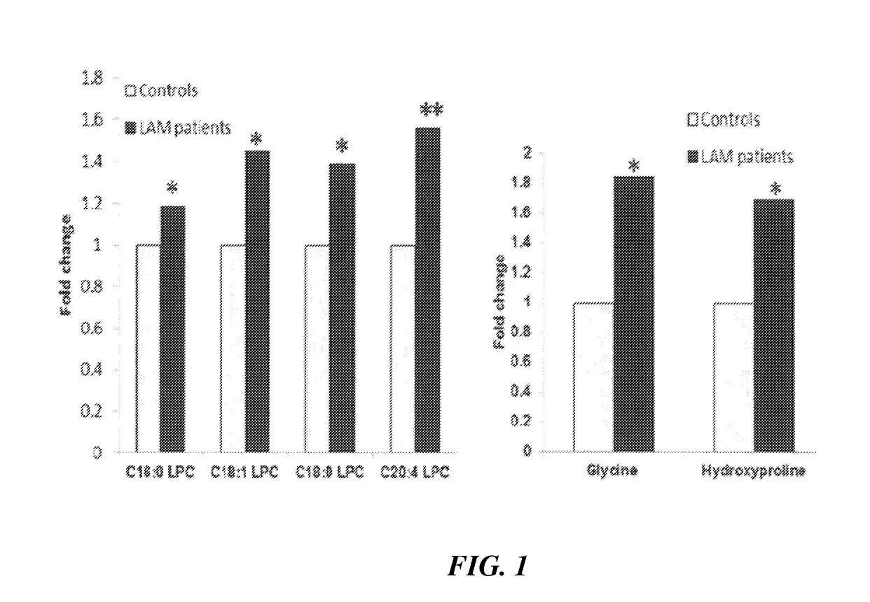

The top significantly altered metabolites included lipid molecules belonging to the phospholipid class. Specifically, four (4) lysophosphatidylcholine (LPC) species (C16:0, C18:0, C18:1 and C20:4 LPC, FIG. 1, left panel), which result from partial hydrolysis of phosphatidylcholines by removal of one of the fatty acid groups, were significantly higher in LAM plasma specimens. (* p<0.05. ** p<0.01). Seven LAM patients' plasma samples were also profiled for polar metabolites, and showed a significant increase in the collagen components glycine and hydroxyproline compared to controls (FIG. 1, right panel).

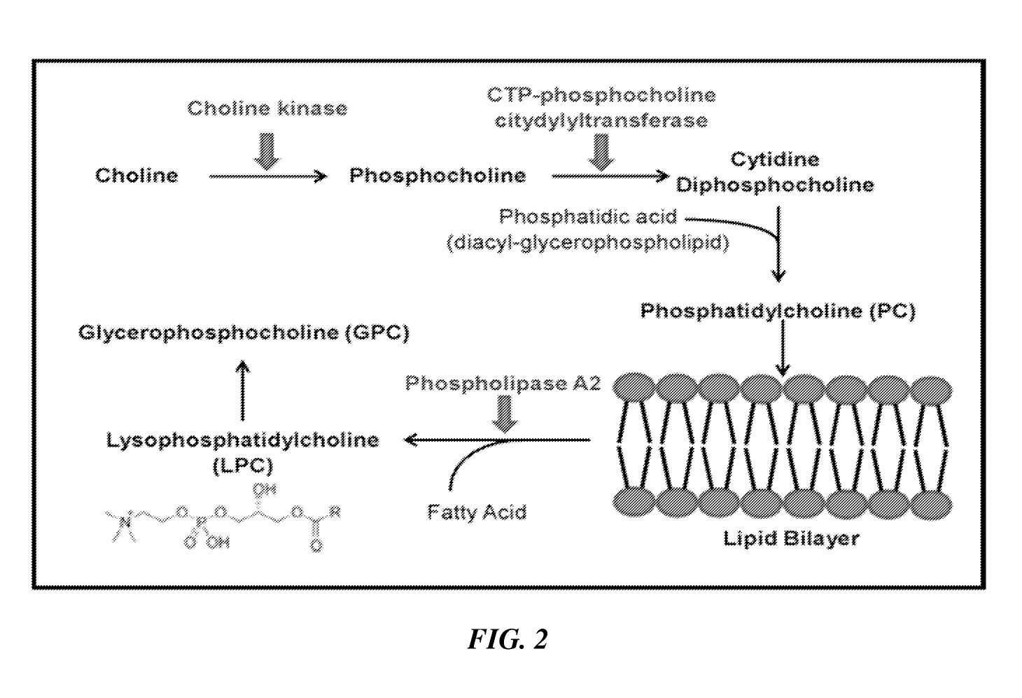

FIG. 2 shows a scheme of the metabolic pathway leading to LPC production. Choline is phosphorylated by choline kinase. Phosphocholine reacts with diacyl-glycerophospholipid phosphatidic acid to form phosphatidylcholine (PC), the main component of cellular membranes. LPCs derive from the hydrolysis of membrane PCs catalyzed by Phospholipase A2, and can be further hydrolyzed to glycerophosphocholine (GPC).

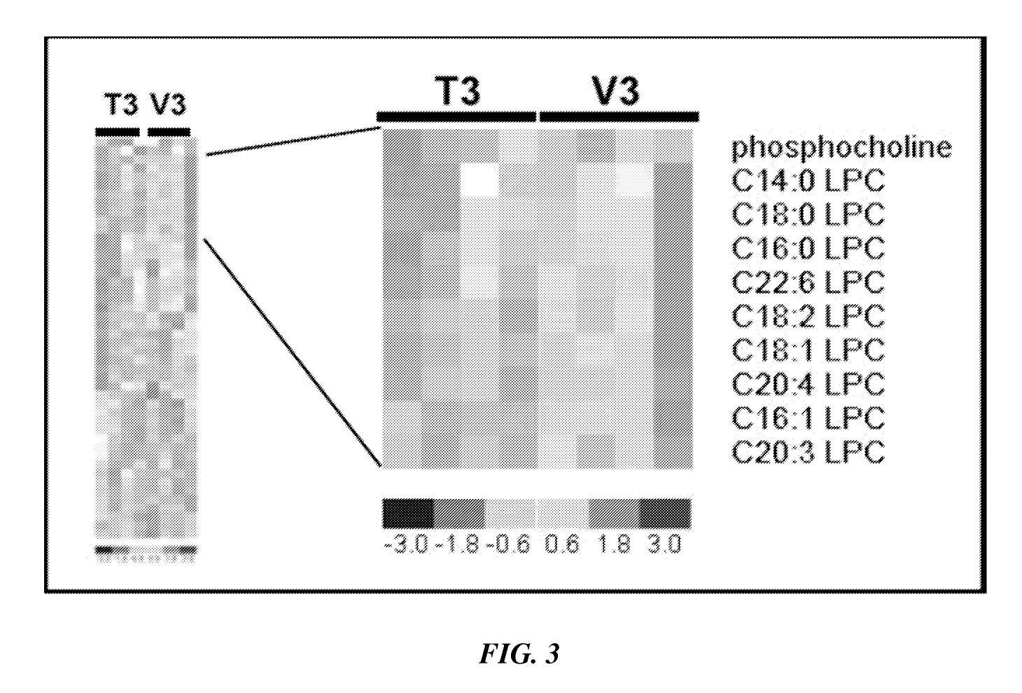

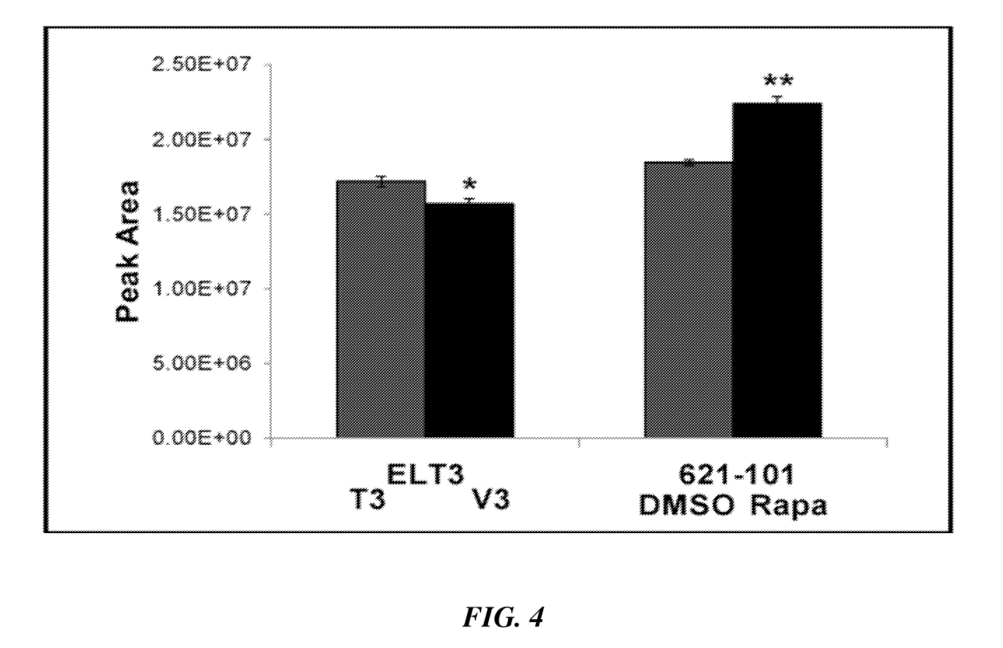

TSC2-deficient cells exhibit dysregulation of choline metabolism. To address the question whether the high levels of circulating LPC in LAM patients are secreted by LAM cells, we performed metabolite profiling of TSC2-deficient rat uterine leiomyoma-derived ELT3 cells (V3; FIG. 3) and their isogenic control re-expressing TSC2 (T3; FIG. 3). Four replicate samples per each group were processed using isopropanol-based lipid extraction and methanol-based polar metabolome purification, followed by LC-MS analysis. Peak areas were quantified. Metabolite normalization (by median per sample) and statistical analysis (two-sample T-Test and False Discovery rate, FDR, as q value) were conducted using MetaboAnalyst. Consistent with patient results, several LPC species, including those found in the plasma profile, were increased significantly in ELT3 cells (V3; FIG. 3) and in their culture media (secreted LPC) (not shown). In addition, we found that phosphocholine was increased significantly in ELT3 cells (V3) vs. control (T3, FIG. 3). Importantly, choline consumption was higher in TSC2-deficient ELT3 cells relative to the TSC2 re-expressing cells, as indicated by the lower levels of choline measured in the conditioned media (q<0.05; FIG. 4). These data indictate that choline metabolism is dysregulated in TSC2-deficient cells.

The references cited herein and throughout the specification are incorporated herein by reference.

1.-84. (canceled)

85. A method of treating lymphangioleiomyomatosis (LAM) or a disease associated with a mutation in a tuberous sclerosis complex (TSC) gene in a subject comprising administering a therapeutically effective amount of a choline kinase (CK) inhibitor and/or a therapeutically effective amount of a phospholipases A2 (PLA2) inhibitor to the subject in need thereof, wherein an elevated level of lysophosphatidylcholine (LPC) in a biological sample from the subject is reduced or is at least 5% closer to a predetermined reference LPC level.

86. The method of claim 85 further comprising diagnosing a subject as having LAM by a method comprising measuring the level of lysophosphatidylcholine (LPC) in a biological sample obtained from the subject, wherein an elevated LPC level in the sample compared to a predetermined reference LPC level is indicative of LAM in the subject prior to administering treatment.

87. The method of claim 85 further comprising monitoring a level of lysophosphatidylcholine (LPC) in a biological sample obtained from the subject prior to treatment or during the course of treatment to determine the course of LAM or the disease in the subject, wherein the LPC level is elevated compared to a predetermined reference level.

88. The method of claim 87 further comprising deciding a course of action for the subject based upon the results of monitoring wherein the course of action is selected from the group consisting of maintained no treatment, terminating current treatment, modify current treatment, and maintained current treatment; and executing the course of action decided.

89. The method of claim 85 further comprising selecting a subject having LAM or a disease associated with a mutation in a TSC gene, wherein the mutation is in the TSC 1 gene and/or TSC 2 gene.

90. The method of claim 89, wherein the mutation results in no protein production or no functional protein.

91. The method of claim 85, wherein the LAM is selected from the group consisting of tuberous sclerosis complex lymphangioleiomyomatosis (TSC-LAM) and non-heritable sporadic form lymphangioleiomyomatosis (S-LAM).

92. The method of claim 87, wherein the predetermined reference LPC level comprises an average LPC level measurement from a plurality of healthy subjects without LAM or any or disease associated with a mutation in a TSC gene, wherein an elevated level of LPC in the sample compared to the predetermined reference LPC level is diagnostic of the presence of LAM or the disease associated with a mutation in a TSC gene respectively.

93. The method of claim 85, wherein the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, P1-103 and cyclophane, and the PLA2 inhibitor is selected from the group consisting of darapladib, bromoenol lactone, varespladib and palmitoyl trifluoromethyl ketone.

94. A method of assessing the efficacy of a drug treatment in a subject, the method comprising:
a. administering to the subject a treatment, wherein the subject has lymphangioleiomyomatosis (LAM) or a disease associated with a mutation in a tuberous sclerosis complex (TSC) gene;
b. measuring the level of lysophosphatidylcholine (LPC) in a first biological sample obtained from the subject at a first time point;
c. measuring the level of LPC in a second biological sample obtained from the subject at a second time point, wherein the first and second biological samples are of the same type, and wherein the first and second time are in chronological order; and
d. comparing the first measurement and the second measurement of LPC wherein the results of the comparison determines whether the drug treatment is effective in the subject.

95. The method of claim 94, wherein optionally, the level of lysophosphatidylcholine (LPC) in a biological sample obtained from the subject was measured prior to the administration of treatment:

96. The method of claim 94 further comprising deciding a course of action for the subject based upon the results of the comparison in step (d) wherein the course of action is selected from the group consisting of maintained current drug application, terminating current drug application, and modify current drug application.

97. The method of claim 94, wherein the treatment is selected from the group consisting of hormone therapy, choline kinase (CK) inhibition therapy, phospholipase A2 (PLA2) inhibition therapy, mTORC1 inhibition therapy, phosphatidylinositol 3-kinase (PI 3-kinase or PI3K) inhibition therapy, oxygen therapy, pleurodesis, embolization, ablation or resection of angiomyolipomas; bronchodilator therapy, withdrawal from estrogen-containing medications, and thoracic duct ligation.

98. The method of claim 97, wherein the CK inhibitor is selected from the group consisting of hemicholinium-3 (HC-3) or HC-3 analogues, bis-quinolinium compounds, acyclic biscationic pyridophane compounds, acyclic biscationic quinolinephane compounds, bispyridinium cyclophanes, 5,5′-dithiobis(2-nitrobenzoic acid), 4′-bispyridyl-5,5′-perfluoroalkyl-2,2′-bisoxazol, 4-chloro-N-methylanilino, 5-Fluorouracil, adenosine, choline analogues, MN58b, TCD828, TCD-717, piperazine, purinyl-6-histamine, N-ethylmaleimide, quinacrine, stearoyl-CoA, CK37, PI-103 and cyclophane.

99. The method of claim 97, wherein the PLA2 inhibitor is selected from the group consisting of darapladib, bromoenol lactone, varespladib and palmitoyl trifluoromethyl ketone.

100. The method of claim 97, wherein the PI3K inhibitor is selected from the group consisting of perifosine, wortmannin, demethoxyviridin, LY294002, CAL101, PX-866, BEZ235, SF1126, INK1117, IPI-145, GDC-0941, BKM120, XL147, XL765, Palomid 529, GSK1059615, ZSTK474, PWT33597, IC87114, TG100-115, CAL263, PI-103, GNE-477, CUDC-907, and AEZS-136.

101. The method of claim 97, wherein the mTORC1 inhibitor is selected from the group consisting of everolimus, temsirolimus and sirolimus.

102. The method of claim 94, wherein the LPC is measured by liquid chromatography coupled to mass spectrometry (LC-MS), enzymatic measurements, or gas chromatography coupled to chemical ionization mass spectrometry (GC-CIMS).

103. The method of claim 94, wherein the LPC measured is selected from the selected from the group consisting of: C14:0 LPC; C16:0 LPC; C16:1 LPC; C18:0 LPC; C18.1 LPC; C18:3 LPC; C18:2 LPC; C20:3 LPC; C20:4 LPC; and C22:6 LPC.

104. The method of claim 94, wherein the biological samples are serum, plasma or urine.

105. A method of treating lymphangioleiomyomatosis (LAM) or a disease associated with a mutation in a tuberous sclerosis complex (TSC) gene in a subject, the method comprising:
a. administering to the subject a treatment;
b. imaging the density and location of carbon-11-labeled choline, fluorine 18-labeled choline or derivatives thereof in the subject at a first time prior to treatment or after the start of treatment;
c. imaging the density and location of carbon-11-labeled choline, fluorine 18-labeled choline or derivatives thereof in the subject at a second time, wherein the first and second time are in chronological order; and
d. comparing the first imaging and the second imaging, wherein the results of the comparison determines the course of LAM or disease in the subject.
